(19) United States
(12) Patent Application Publication
(10) Pub. No.: US 2003/0180797 A1
(43) Pub. Date: Sep. 25, 2003

(54) Identification of ligands for a receptor family and related methods

(76) Inventors: Lin Yu, San Diego, CA (US); Qing Dong, San Diego, CA (US); Fabrice Pierre, La Jolla, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 10/103,535

(22) Filed: Mar. 20, 2002

(51) Int. Cl. G01N 33/53; C12Q 1/48; G01N 33/543

(52) U.S. Cl. 435/7.1; 436/518

(57) ABSTRACT

The invention provides a method of identifying a population of bi-ligands to receptors in a receptor family. The method can include the steps of generating a first population of molecules comprising a specificity ligand having binding activity for a receptor in a receptor family, the specificity ligand attached to a first plurality of chemical moieties at a position on the specificity ligand to direct the specificity ligand to a specificity site and the chemical moieties to a conserved site of the receptor; screening the population of molecules for binding to the receptor; and identifying a bi-ligand having increased binding activity for the receptor relative to the specificity ligand alone, thereby identifying a common ligand having binding activity for the receptor. The method can further include the steps of generating a second population of molecules comprising the common ligand attached to a second plurality of chemical moieties.

### BACKGROUND OF THE INVENTION

### Field of the Invention

The present invention relates generally to drug discovery methods and more specifically to methods of generating libraries of biligand drugs.

Two general approaches have traditionally been used for drug discovery: screening for lead compounds and structure-based drug design. Both approaches have advantages and disadvantages, with the most significant disadvantage being the laborious and time-consuming nature of using these approaches to discover new drugs.

Drug discovery and development based on screening for lead compounds involves generating a pool of candidate compounds, often using combinatorial chemistry in which compounds are synthesized by combining chemical groups to generate a large number of diverse candidate compounds that bind to the target or that inhibit binding to the target. The candidate compounds are screened with a drug target of interest to identify lead compounds that bind to the target or inhibit binding to the target. However, the screening process to identify a lead compound can be laborious and time consuming.

Structure-based drug design is an alternative approach to identifying candidate drugs. Structure-based drug design uses three-dimensional structural data of the drug target as a template to model compounds that bind to the drug target and alter its activity. The compounds identified as potential drug candidates using structural modeling are used as lead compounds for the development of candidate drugs that exhibit a desired activity toward the drug target.

Identifying compounds using structure-based drug design can be advantageous over the screening approach in that modifications to the compound can often be predicted based on the modeling studies. However, obtaining structures of relevant drug targets and of drug targets complexed with test compounds is extremely time consuming and laborious, often taking years to accomplish. The long time period required to obtain structural information useful for developing drug candidates is particularly limiting with regard to the growing number of newly discovered genes, which are potential drug targets, identified in genomics studies.

Despite the time-consuming and laborious nature of these approaches to drug discovery, both screening for lead compounds and structure-based drug design have led to the identification of a number of useful drugs, such as receptor agonists and antagonists. However, even with drugs useful for treating particular diseases, many of the drugs can have unwanted side effects. For example, in addition to binding to the drug target in a pathogenic organism or cancer cell, in some cases the drug also binds to an analogous protein in the patient being treated with the drug, which can result in unwanted side effects. Therefore, drugs that have high affinity and specificity for a target are particularly useful because administration of a more specific drug at lower dosages will minimize unwanted side effects.

In addition to undesirable side effects of a drug, a number of drugs that were previously highly effective for treating certain diseases have become less effective during prolonged clinical use due to the development of resistance. Drug resistance has become increasingly problematic, particularly with regard to administration of antibiotics. A number of pathogenic organisms have become resistant to several drugs due to prolonged clinical use and, in some cases, have become almost totally resistant to currently available drugs. Furthermore, certain types of cancer develop resistance to cancer therapeutic agents. Therefore, drugs that are refractile to the development of resistance would be particularly desirable for treatment of a variety of diseases.

One approach to developing such drugs is to find compounds that bind to a target protein such as a receptor or enzyme. When such a target protein has two adjacent binding sites, it is especially useful to find “biligand” drugs that can bind at both sites simultaneously. However, the rapid identification of biligand drugs having a high binding affinity has been difficult. Biligand drug candidates have been identified using rational drug design, but previous methods are time-consuming and require a precise knowledge of structural features. Recent advances in nuclear magnetic spectroscopy (NMR) have allowed the determination of the three-dimensional interactions between a ligand and an receptor in a few instances. However, these efforts have been limited by the size of the receptor and can take years to map and analyze the complete structure of the complexes of receptor and ligand.

Thus, there exists a need to rapidly and efficiently identify compounds that bind to a drug target with improved affinity and/or specificity. The present invention satisfies this need and provides related advantages as well.

### SUMMARY OF THE INVENTION

The invention provides a method of identifying a population of bi-ligands to receptors in a receptor family. The method can include the steps of generating a first population of molecules comprising a specificity ligand having binding activity for a receptor in a receptor family, the specificity ligand attached to a first plurality of chemical moieties at a position on the specificity ligand to direct the specificity ligand to a specificity site and the chemical moieties to a conserved site of the receptor; screening the population of molecules for binding to the receptor; and identifying a bi-ligand having increased binding activity for the receptor relative to the specificity ligand alone, thereby identifying a common ligand having binding activity for the receptor. The method can further include the steps of generating a second population of molecules comprising the common ligand attached to a second plurality of chemical moieties.

### BRIEF DESCRIPTION OF THE DRAWINGS

FIG. 1 shows a diagram representing bi-ligands bound to various receptors. The bi-ligand depicted contains three components, a common ligand, a specificity ligand and an expansion linker. FIG. 1A shows a population of bi-ligands sharing the same specificity ligand, depicted as a triangle, and various chemical moieties, depicted as a circle, pentagon, rectangle and hexagon, attached via an expansion linker (indicated by two lines). Only the bi-ligand having a pentagon contains a common ligand that allows binding to the conserved site of receptor 1. FIG. 1B shows a population of bi-ligands sharing the same common ligand, depicted as a pentagon, and various chemical moieties, depicted as a triangle, square, circle and star, attached via an expansion linker. The specificity ligand binds to a specificity site on the receptor and is depicted as a triangle, square, circle and star for bi-ligands that bind to receptors 1 through 4, respectively. The expansion linker, indicated by two lines, bridges the common ligand and specificity ligand in an orientation allowing both the common ligand and specificity ligand to bind simultaneously to the respective conserved site and specificity site on the receptor.

FIG. 2 shows a flow diagram depicting the identification of a common ligand, also called a common ligand mimic (CLM). A specificity ligand (SL) is used to build a library of compounds at a desired attachment point. The library is screened for a bi-ligand exhibiting increased binding activity to a receptor relative to the specificity ligand alone. Such a bi-ligand allows the identification of a common ligand (CLM′).

FIG. 3 shows a reaction scheme for the synthesis of a pyridine dicarboxylate derivative.

FIG. 4 shows a reaction scheme for the synthesis of rhodanine and thiazolidinedione-based bi-ligand inhibitors.

FIG. 5 shows a reaction scheme for the synthesis of pseudothiohydantoin-based bi-ligand inhibitors.

FIG. 6 shows a reaction scheme for the synthesis of benzimidazole-based bi-ligand inhibitors.

FIG. 7 shows a reaction scheme for the synthesis of pyridine napthalene-based bi-ligand inhibitors.

FIG. 8 shows the activity of various bi-ligands for binding to dihydrodipicolinate reductase (DHPR).

FIG. 9 shows the structures of a variety of rhodanine derivatives and their binding activities (IC₅₀).

FIG. 10 shows the structures of various thiazolidinedione derivatives and their binding activities (IC₅₀).

FIG. 11 shows the structure and binding activity of various thiazolidinedione analogs to various members of an oxidoreductase receptor family. The values shown are IC₅₀ values.

FIG. 12 shows structures and activity of derivatives of a pseudothiohydantoin derivative. The values shown are IC₅₀ values.

FIG. 13 shows the structure and binding activity of various psuedothiohydantoin analogs to various members of an oxidoreductase receptor family. The values shown are IC₅₀ values.

FIG. 14 shows the structures of a variety of benzimidazole analogs and their binding activities (IC₅₀).

### DETAILED DESCRIPTION OF THE INVENTION

The invention provides methods for identifying common ligands for a receptor family, which can be used to build libraries of molecules that can be screened for binding to a variety of receptors in a receptor family. The methods are applicable to the identification of ligands that bind with improved affinity and/or specificity to a desired target receptor. The methods of the invention are advantageously used to develop bi-ligands that bind to two sites on a receptor, a common site and a specificity site, and can have optimized binding characteristics. By using a bi-ligand that binds to two sites, individual ligands that bind to either a common site or specificity site are combined to exploit the binding of the individual ligands in a synergistic manner. The methods of the invention provide for the independent optimization of the common ligand and specificity ligand of a bi-ligand, allowing more rapid identification of compounds having improved binding characteristics and that can function as drugs.

The methods of the invention allow the generation of libraries of compounds that share a common ligand and therefore can be screened for binding activity to various members of a receptor family. Such libraries allow more efficient identification of ligands for a receptor in a receptor family since the same library or similar libraries can be used repeatedly for different members of the receptor family. Such libraries can be particularly useful for identifying ligands for receptor family members newly identified from genomics studies. The methods of the invention allow the independent optimization of portions of a bi-ligand molecule. The optimization of binding characteristics of a portion of a bi-ligand molecule provide for increased diversity of a library while simultaneously focusing a library on a particular receptor family or particular member of a receptor family.

The methods of the invention can be used to identify a common ligand that binds to receptors in a target receptor family. In some cases, a common ligand to a receptor family is already known. For example, NAD is a natural common ligand for dehydrogenases, and ATP is a natural common ligand for kinases. In addition to naturally occurring substrates and cofactors, analogs or variants of these substrates and cofactors that bind to a conserved site are also often known. However, natural common ligands such as coenzymes and cofactors and known derivatives thereof often have limitations regarding their usefulness as a starting compound. Substrates and cofactors often undergo a chemical reaction, for example, transfer of a group to another substrate or reduction or oxidation during the enzymatic reaction. However, it is desirable that a ligand to be used as a drug is not metabolizable. The methods of the invention are advantageous in that a common ligand having a desirable property, for example, not metalizable, can be identified more efficiently. In addition, the methods of the invention can be used to identify a common ligand having optimized binding properties such as increased binding affinity, increased specificity for a subfamily of receptors in a receptor family, improved biological activity and/or improved pharmacological property such as improved absorption, distribution, metabolism and/or elimination (ADME).

As used herein, the term “ligand” refers to a molecule that can selectively bind to a receptor. The term “selectively” means that the binding interaction is detectable over non-specific interactions as measured by a quantifiable assay. A ligand can be essentially any type of molecule such as an amino acid, peptide, polypeptide, nucleic acid, carbohydrate, lipid, or small organic compound. The term ligand refers both to a molecule capable of binding to a receptor and to a portion of such a molecule, if that portion of the molecule is capable of binding to the receptor. For example, a bi-ligand, which contains a common ligand and specificity ligand, is considered a ligand, as would the common ligand and specificity ligand portions since they can bind to a common site and specificity site, respectively. As used herein, the term “ligand” excludes a single atom, for example, a metal atom. Derivatives, analogues and mimetic compounds are also intended to be included within the definition of this term, including the addition of metals or other inorganic molecules, so long as the metal or inorganic molecule is covalently attached to the ligand such that the dissociation constant of the metal from the ligand is less than 10⁻¹⁴ M. A ligand can be multi-partite, comprising multiple ligands capable of binding to different sites on one or more receptors, such as a bi-ligand. The ligand components of a multi-partite ligand can be joined together by an expansion linker.

As used herein, the term “common ligand” refers to a ligand that binds to a conserved site on receptors in a receptor family. A “common ligand mimic” (CLM) refers to a common ligand that has structural and/or functional similarities to a natural common ligand but is not naturally occurring. Thus, a common ligand mimic can be a modified natural common ligand, for example, an analogue or derivative of a natural common ligand, and is considered to be a common ligand. As used herein, “natural common ligand” refers to a ligand that is found in nature and binds to a common site on receptors in a receptor family. It is understood that a common ligand need not bind to all members of a receptor family but does bind to at least two members of a receptor family and can bind to several, most, or all members of the receptor family.

In many cases, an identified receptor family will have a natural common ligand that is already known. For example, it is known that dehydrogenases bind to dinucleotides such as NAD or NADP. Therefore, NAD or NADP are natural common ligands to a number of dehydrogenase family members. Similarly, kinases bind ATP, which is therefore a natural common ligand to kinases. Other natural common ligands of a receptor family can be coenzymes cofactors or substrates that function in an enzyme reaction. Exemplary cofactor natural common ligands include, for example, nicotinamide adenine dinucleotide (NAD) and nicotinamide adenine dinucleotide phosphate (NADP), thiamine pyrophosphate, flavin adenine dinucleotide (FAD) and flavin mononucleotide (FMN), pyridoxal phosphate, coenzyme A, tetrahydrofolate, adenosine triphosphate (ATP), guanosine triphosphate (GTP), S-adenosyl methionine (SAM), isoprenyl groups such as farnesyl and geranyl, dihydropterin, biotin, heme or other porphyrins, and the like. Reduced and/or oxidized forms or other naturally occurring derivatives of these cofactors are also included as exemplary natural common ligands so long as such forms bind to a receptor.

As used herein, the term “specificity ligand” refers to a ligand that binds to a specificity site on a receptor. A specificity ligand can bind to a specificity site as an isolated molecule or can bind to a specificity site when attached to a common ligand, as in a bi-ligand. When a specificity ligand is part of a bi-ligand, the specificity ligand can bind to a specificity site that is proximal to a conserved site on a receptor. A “natural specificity ligand” refers to a ligand that is found in nature and binds to a specificity site of a receptor. In the case of an enzyme, a natural specificity ligand is a substrate specific to the enzyme, for example, lactate for lactate dehydrogenase, and the like.

As used herein, the term “bi-ligand” refers to a ligand comprising two covalently linked ligands, a common ligand and a specificity ligand, which can be tethered by an expansion linker. Each of the two ligand components of a bi-ligand bind to independent sites on a receptor. The common ligand and specificity ligand of a bi-ligand are positioned so that the common ligand and specificity ligand can simultaneously bind to the conserved site and specificity site, respectively, of a receptor in a receptor family.

As used herein, the term “expansion linker” refers to a chemical group that links two ligands that bind to the same receptor. An expansion linker is used to bridge a common ligand to one or more specificity ligands. An expansion linker can be optimized to provide positioning and orientation of the specificity ligand relative to the common ligand such that the common ligand and specificity ligand are positioned to bind to their respective conserved site and specificity site on a receptor.

As used herein, the term “conserved site” on a receptor” refers to a site that has structural and/or functional characteristics common to members of a receptor family. A conserved site contains amino acid residues sufficient for activity and/or function of the receptor that are accessible to binding of a natural common ligand. For example, the amino acid residues sufficient for activity and/or function of a receptor that is an enzyme can be amino acid residues in a substrate binding site of the enzyme. For example, the conserved site in an enzyme that binds a cofactor or coenzyme can be amino acid residues that bind the cofactor or coenzyme. Accordingly, a conserved site in an oxidoreductase binds to NADH and/or NADPH, and a conserved site in a kinase binds ATP.

As used herein, the term “specificity site” refers to a site on a receptor that provides the binding site for a ligand exhibiting specificity for a receptor. A specificity site on a receptor imparts molecular properties that distinguish the receptor from other receptors in the same receptor family. For example, if the receptor is an enzyme, the specificity site can be a substrate binding site that distinguishes two members of a receptor family that exhibit substrate specificity. A substrate specificity site can be exploited as a potential binding site for the identification of a ligand that has specificity for one receptor over another member of the same receptor family. For example, the lactate dehydrogenase substrate binding site that binds lactate is a specificity site for lactate dehydrogenase. Similarly, the β-hydroxy-β-methylglutaryl (HMG) CoA reductuase substrate binding site that binds HMG CoA is a specificity site for HMG CoA reductase. A specificity site is distinct from a conserved site in that a natural common ligand does not bind to a specificity site.

As used herein, the term “receptor” refers to a polypeptide that is capable of selectively binding a ligand. The function and/or activity of a receptor can be enzymatic activity or ligand binding. Receptors can include, for example, enzymes such as kinases, oxidoreductases such as dehydrogenases, GTPases, carboxyl transferases, acyl transferases, decarboxylases, transaminases, racemases, methyl transferases, formyl transferases, α-ketodecarboxylases, isoprenyltransferases, and the like.

Furthermore, the receptor can be a functional fragment or modified form of the entire polypeptide so long as the receptor exhibits selective binding to a ligand. A functional fragment of a receptor is a fragment exhibiting binding to a common ligand and a specificity ligand. As used herein, the term “enzyme” refers to a molecule that carries out a catalytic reaction by converting a substrate to a product.

Enzymes can be classified based on Enzyme Commission (EC) nomenclature recommended by the Nomenclature Committee of the International Union of Biochemistry and Molecular Biology (IUBMB)(see, for example, www.expasy.ch/sprot/enzyme.html). For example, oxidoreductases are classified as oxidoreductases acting on the CH—OH group of donors with NAD⁺ or NADP⁺ as an acceptor (EC 1.1.1); oxidoreductases acting on the aldehyde or oxo group of donors with NAD⁺ or NADP⁺ as an acceptor (EC 1.2.1); oxidoreductases acting on the CH—CH group of donors with NAD⁺ or NADP⁺ as an acceptor (EC 1.3.1); oxidoreductases acting on the CH—NH₂ group of donors with NAD⁺ or NADP⁺ as an acceptor (EC 1.4.1); oxidoreductases acting on the CH—NH group of donors with NAD⁺ or NADP⁺ as an acceptor (EC 1.5.1); oxidoreductases acting on NADH or NADPH (EC 1.6); and oxidoreductases acting on NADH or NADPH with NAD⁺ or NADP⁺ as an acceptor (EC 1.6.1).

Additional oxidoreductases include oxidoreductases acting on a sulfur group of donors with NAD⁺ or NADP⁺ as an acceptor (EC 1.8.1); oxidoreductases acting on diphenols and related substances as donors with NAD⁺ or NADP⁺ as an acceptor (EC 1.10.1); oxidoreductases acting on hydrogen as donor with NAD⁺ or NADP⁺ as an acceptor (EC 1.12.1); oxidoreductases acting on paired donors with incorporation of molecular oxygen with NADH or NADPH as one donor and incorporation of two atoms (EC 1.14.12) and with NADH or NADPH as one donor and incorporation of one atom (EC 1.14.13); oxidoreductases oxidizing metal ions with NAD⁺ or NADP⁺ as an acceptor (EC 1.16.1); oxidoreductases acting on —CH₂ groups with NAD⁺ or NADP⁺ as an acceptor (EC 1.17.1); and oxidoreductases acting on reduced ferredoxin as donor, with NAD⁺ or NADP⁺ as an acceptor (EC 1.18.1).

Other enzymes include transferases classified as transferases transferring one-carbon groups (EC 2.1); methyltransferases (EC 2.1.1); hydroxymethyl-, formyl- and related transferases (EC 2.1.2); carboxyl- and carbamoyltransferases (EC 2.1.3); acyltransferases (EC 2.3); and transaminases (EC 2.6.1). Additional enzymes include phosphotransferases such as phosphotransferases transferring phosphorous-containing groups with an alcohol as an acceptor (kinases) (EC 2.7.1); phosphotransferases with a carboxyl group as an acceptor (EC 2.7.2); phosphotransfer with a nitrogenous group as an acceptor (EC 2.7.3); phosphotransferases with a phosphate group as an acceptor (EC 2.7.4); and diphosphotransferases (EC 2.7.6).

Enzymes can also bind coenzymes or cofactors such as nicotinamide adenine dinucleotide (NAD) and nicotinamide adenine dinucleotide phosphate (NADP), thiamine pyrophosphate, flavin adenine dinucleotide (FAD) and flavin mononucleotide (FMN), pyridoxal phosphate, coenzyme A, and tetrahydrofolate or other cofactors or substrates such as adenosine triphosphate (ATP), guanosine triphosphate (GTP), S-adenosyl methionine (SAM), isoprenyl groups such as farnesyl and geranyl, dihydropterin, biotin, heme or other porphyrins, and the like. In addition, enzymes that bind newly identified cofactors or enzymes can also be receptors.

As used herein, the term “receptor family” refers to a group of two or more receptors that bind a natural common ligand. Members of a receptor family generally contain a conserved amino acid motif because certain amino acid residues, or amino acids having similar physicochemical characteristics, are required for the structure, function and/or activity of the receptor and are therefore conserved between members of the receptor family. Methods of identifying related members of a receptor family are well known to those skilled in the art and include sequence alignment algorithms and identification of conserved patterns or motifs in a group of polypeptides, which are described in more detail below. Members of a receptor family can also be identified by determining binding to a natural common ligand. Accordingly, a receptor predicted to be a member of a receptor family can be confirmed by determining if the receptor binds a natural common ligand.

As used herein, the term “population” refers to a group of two or more different molecules. A population can be as large as the number of individual molecules currently available to the user or which can be made by one skilled in the art. A population can be as small as two molecules and as large as 10¹⁰ molecules. A population can contain two or more, three or more, five or more, seven of more, ten or more, twelve or more, fifteen or more, or twenty of more different molecules. A population can also contain tens or hundreds of different molecules or even thousands of different molecules. For example, a population can contain about 20 to 100,000 different molecules or more, for example, about 25 or more, about 30 or more, about 40 or more, about 50 or more, about 75 or more, about 100 or more, about 150 or more, about 200 or more, about 300 or more, about 500 or more, about 1000 or more, and even about 10,000 or more different molecules. A population of bi-ligands is derived, for example, by chemical synthesis and is substantially free of naturally occurring substances.

As used herein, the term “library” refers to an intentionally created set of differing molecules. A library generally contains a population of about 100 or more different molecules. For example, a library can contain about 150 or more, about 200 or more, about 250 or more, about 300 or more, about 400 or more, about 500 or more, about 700 or more, about 1000 or more, about 1500, about 2000 or more, about 3000 or more, about 5000 or more, about 10,000 or more, about 20,000 or more, about 30,000 or more, about 50,000 or more, about 100,000 or more or even about 1×10⁶ or more different molecules.

As used herein, the term “specificity” refers to the ability of a ligand to differentially bind to one receptor over another receptor in the same receptor family. The differential binding of a particular ligand to a receptor is measurably higher than the binding of the ligand to at least one other receptor in the same receptor family. A ligand having specificity for a receptor refers to a ligand exhibiting specific binding that is at least two-fold higher for one receptor over another receptor in the same receptor family. Specificity can also be exhibited over 2 or more other members of the receptor family, 3 or more, 4 or more, 5 or more, 6 or more, 7 or more, 8 or more, 9 or more, or even 10 or more other members of the receptor family, or specificity can be exhibited by essentially all of the receptors in a receptor family.

A ligand having specificity will have 2-fold higher affinity or greater, and can have about 3-fold, 4-fold, 5-fold, 10-fold, 15-fold, 20-fold, 30-fold, 50-fold, 100-fold, 200-fold, 300-fold, 400-fold, 500-fold, 1000-fold, 1500-fold, 2000-fold, 5000-fold, 10,000-fold, 20,000-fold, 50,000-fold, 100,000-fold or even 1×10⁶-fold or higher affinity or greater. Also, a ligand can have specificity for one receptor over 2 other members, 3 other members, 4 other members, 5 other members, 6 other members, 7 other members, 8 other members, 9 other members, 10 other members, 15 other members, 20 other members, or even essentially all other members of a receptor family. However, it is not necessary to show specificity for one receptor over all other members of the receptor family but, rather, it is sufficient to show that a ligand has specificity for a receptor relative to at least one other member of the receptor family.

When referring to a population of bi-ligands, the population can contain a bi-ligand having specificity for at least one receptor over another receptor in the same receptor family and can contain two or more bi-ligands, each of which has specificity for two different receptors in the receptor family. Thus, a population of bi-ligands can contain 3 or more bi-ligands, 4 or more bi-ligands, 5 or more bi-ligands, 6 or more bi-ligands, 7 or more bi-ligands, 8 or more bi-ligands, 9 or more bi-ligands, 10 or more bi-ligands, 15 or more bi-ligands, or even 20 or more bi-ligands, each of which has specificity for a different receptor in the same receptor family.

The invention provides a method for identifying a common ligand for a receptor family. The method includes the steps of generating a population of molecules comprising a specificity ligand having binding activity for a receptor in a receptor family, the specificity ligand attached to a plurality of chemical moieties at a position on the specificity ligand to direct the specificity ligand to a specificity site and the chemical moieties to a conserved site of the receptor; screening the population of molecules for binding to the receptor; and identifying a bi-ligand having increased binding activity for the receptor relative to the specificity ligand alone, thereby identifying a common ligand having binding activity for the receptor. Such a common ligand can be used to generate a bi-ligand having specificity for a receptor in a receptor family, as described in more detail below.

The methods of the invention are used to identify a common ligand for a receptor family. A common ligand is identified by tethering a plurality of chemical moieties to a specificity ligand to build a population or library of bi-ligands, which can be screened for desirable binding characteristics. Methods for generating a population of bi-ligands using a common ligand and diverse chemical moieties that can be screened for binding to a specificity site have been described previously (see U.S. Pat. No. 6,333,149, issued Dec. 25, 2001; WO 99/60404; and WO 00/75364, each of which is incorporated herein by reference). Similar methods can be used to identify a common ligand by making bi-ligands containing a specificity ligand and diverse chemical moieties that can be screened for binding to a conserved site of a receptor. The combination of two ligands into a single bi-ligand results in a synergistic increase in binding activity over that of a specificity ligand and common ligand alone. By tethering potential common ligands to a specificity ligand to generate a bi-ligand, the binding activity of various common ligand structures can be tested even if the binding activity of the isolated common ligand has relative modest binding affinity. Thus, the methods can be used to readily identify a variety of common ligands suitable for generating a diverse library of bi-ligands that can be screened against various members of a family of receptors.

The methods of the invention are used to identify a common ligand for a receptor in a receptor family. Such a common ligand can be used to develop bi-ligands, which bind to two independent sites on a receptor. The combination of two ligands into a single molecule allows both ligands to simultaneously bind to a receptor and thus can provide synergistically higher affinity than either ligand alone (Dempsey and Snell, Biochemistry 2:1414-1419 (1963); and Radzicka and Wolfenden, Methods Enzymol. 249:284-303 (1995), each of which is incorporated herein by reference). The generation of populations and libraries of bi-ligands focused for binding to a receptor family or particular receptor in a receptor family has been described previously as (see WO 99/60404, which is incorporated herein by reference). The present invention provides methods for increasing the diversity of bi-ligand libraries while simultaneously preserving the ability to focus a library for binding to a receptor family.

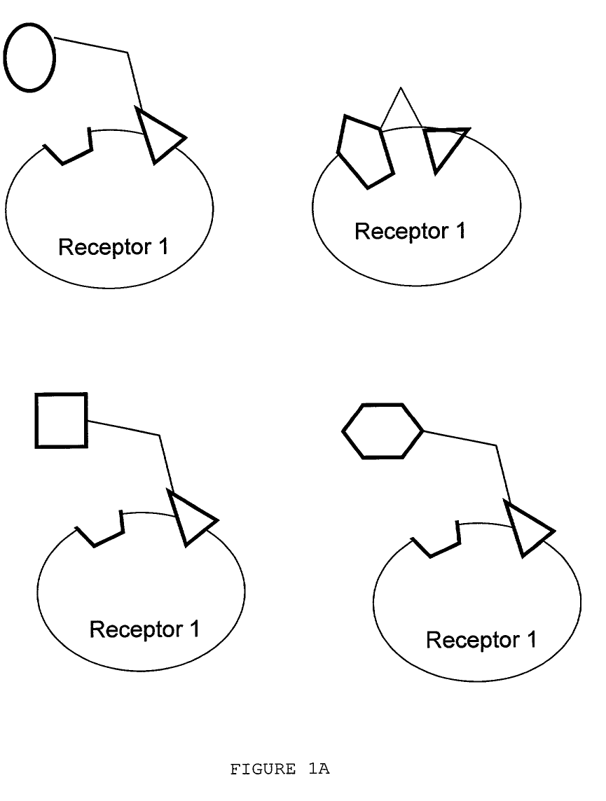

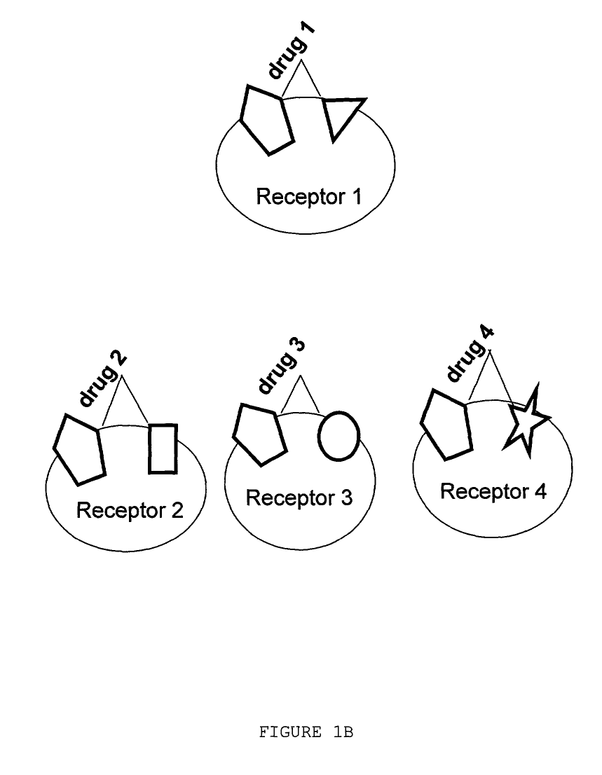

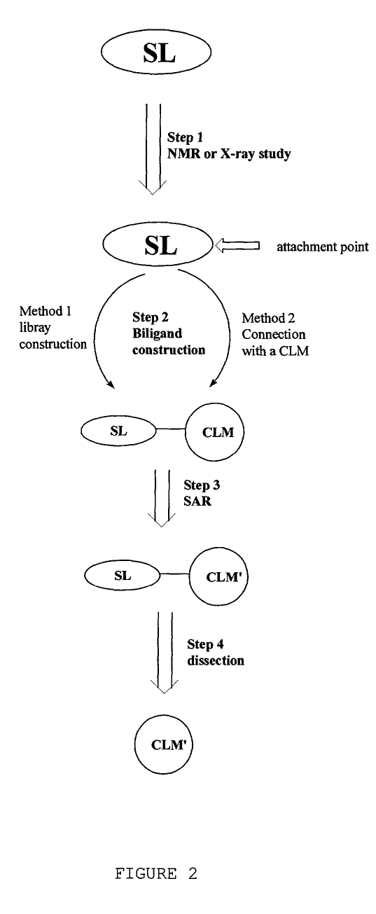

As depicted in FIG. 1A, a specificity ligand (triangle) is shown bound to the specificity site of receptor 1. Various chemical moieties, depicted as a pentagon, circle, rectangle and hexagon, are attached to the specificity ligand via an expansion linker. Only the common ligand depicted as a pentagon can bind to the conserved site of receptor 1. Thus, screening such a population of molecules allows the identification of a common ligand that binds to a conserved site of a receptor. Once such, a common ligand has been identified, a plurality of chemical moieties is attached to the common ligand in a position to bind the specificity site of a receptor. A population containing the common ligand and various chemical moieties can be screened for binding to one or more receptors in a receptor family (see FIG. 1B). Such a population of molecules can be used repeatedly to screen for binding of various receptors in a receptor family that binds the common ligand. FIG. 2 shows a schematic diagram of the identification of a common ligand using methods of the invention.

The methods of the invention can be used to generate a population of bi-ligands while independently increasing diversity and/or optimizing a portion of a bi-ligand molecule. To generate a bi-ligand, a common ligand is identified that binds to multiple members of a receptor family. It is understood that the identified common ligand need not bind to all members of a receptor family so long as the common ligand binds to at least two members of a receptor family. A common ligand can bind to a subfamily of a receptor family, for example, a pharmacophore family that binds a ligand in a particular conformation (see U.S. application Ser. No. 09/747,174, which is incorporated herein by reference). Thus, by screening for a common ligand and thereby providing increased diversity of a bi-ligand library, a library of bi-ligands can be focused to optimize binding to a receptor subfamily that have more similar binding properties than the receptor family as a whole.

When developing bi-ligands having binding activity for a receptor family, it is generally desirable to use a common ligand having relatively modest binding activity, for example, mM to μM binding activity. Since the common ligand binds to multiple members of a receptor family, a high affinity common ligand could bind to other members of a receptor family in addition to the target receptor. It is therefore desirable to identify common ligands having modest affinity, generally at or below the affinity of the natural common ligand that binds to the same conserved site. Generally, modest affinity ligands will have affinity for a receptor of about 10⁻² to 10⁻⁷ M, particularly about 10⁻³ to 10⁻⁶ M, for example, about 10⁻³, about 10⁻⁴, about 10⁻⁵ or about 10⁻⁶.

The binding activity of a bi-ligand is increased relative to the common ligand or specificity ligand individually. Although modest binding activity of an isolated common ligand is desirable, such modest binding activity of a common ligand can be difficult to measure when the common ligand is isolated. It can therefore be more difficult to identify an isolated common ligand having the desired property of modest binding affinity since it is more difficult to measure binding of ligands exhibiting modest binding affinity. Furthermore, modifying a common ligand to identify an alternative chemical form or optimized binding property can also be difficult to identify if the common ligand has modest binding affinity. By tethering potential common ligands or common ligand variants to a specificity ligand, the methods allow more efficient screening and identification of a common ligand or a common ligand variant having improved binding characteristics since the specificity ligand provides increased overall binding activity, resulting in more rapid and efficient identification of a common ligand, which could be difficult or essentially impossible to identify using other methods.

The use of a common ligand that is a mimic of a natural common ligand can be advantageous because natural common ligands can be more effective in crossing biological membranes such as bacterial or eukaryotic cell membranes. For example, a transport system actively transports the nicotinamide mononucleotide half of the NAD molecule (Zhu et al., J. Bacteriol. 173:1311-1320 (1991)). Therefore, it is possible that a bi-ligand comprising a common ligand, or derivative thereof, that is actively transported into a cell will facilitate the transport of the bi-ligand across the membrane. Facilitating the transport of a bi-ligand across the membrane overcomes one of the major limitations to the effectiveness of new drug candidates the ability of the drug candidate to cross the membrane. The methods of the invention allow the identification of mimics of a natural common ligand exhibiting more efficient membrane penetration of other desirable properties.

Additionally, the common ligand is used as a platform to attach specificity ligands capable of binding to a specificity site of a receptor. This requires that the common ligand and specificity ligand be oriented for optimized binding to the conserved site and specificity site. However, the position on a natural common ligand that is oriented towards a specificity site is not always readily derivatizable for attaching a chemical group. Finally, some substrates or cofactors are highly charged, often making them less able to cross the membrane to target a receptor inside the cell. Therefore, it is often desirable to identify additional common ligands that are useful for generating bi-ligands.

To identify a common ligand using methods of the invention, a specificity ligand is selected that binds to a desired target receptor. A target receptor is selected based on desired characteristics such as being a member of a particular receptor family and being expressible in quantities suitable for required screening assays, as disclosed herein. Methods of identifying a specificity ligand for the target receptor are well known to those skilled in the art. For example, a specificity ligand can be identified by screening a variety of compounds for competitive binding with a known specificity ligand. Such a known specificity ligand can be, for example, a substrate for an enzyme receptor such as lactate for lactate dehydrogenase, and the like. In addition, a specificity ligand can be screened and identified using analogs or derivatives of a known specificity ligand. A specificity ligand can also be identified by screening a population of bi-ligands containing a common ligand and variable chemical moieties for binding activity to a receptor in a receptor family (U.S. Pat. No. 6,333,149; WO 99/60404; and WO 00/75364). A bi-ligand that binds to a specificity site and has specificity for a receptor contains a specificity ligand for that receptor. The binding activity of a selected specificity ligand to a specificity site of a receptor can be confirmed by competitive binding with a known specificity ligand for the target receptor.

A specificity ligand binds to a specificity site of a receptor. It is understood that a specificity ligand need not bind only to a single receptor in a receptor family but can bind to more than one receptor in a receptor family so long as the specificity ligand binds to the specificity sites of the receptors in the receptor family. A specificity ligand that binds to multiple members of a receptor family is distinct from a common ligand

A plurality of chemical moieties is attached to the specificity ligand. Methods for generating a plurality of chemical moieties and attaching them to a specificity ligand are well known to those skilled in the art, as described in more detail below. The chemical moieties are attached to the specificity ligand so that the specificity ligand and the plurality of chemical moieties can simultaneously bind to a specificity site and conserved site, respectively, of a target receptor.

To attach the plurality of chemical moieties to the specificity ligand so that the chemical moieties are positioned for binding to the conserved site of the receptor, an appropriate position on the specificity ligand for attaching the chemical moieties is determined. Such a position for attaching chemical moieties is identified by determining which atoms on the specificity ligand are proximal to the conserved site of a receptor when the specificity ligand is bound to the receptor. These atoms on the specificity ligand are identified by determining which atoms of a receptor are at the interface of the conserved site and specificity site and which atoms of a ligand are proximal to the interface atoms when the ligand is bound to the receptor.

Methods of determining an appropriate position on a specificity ligand or common ligand to attach the plurality chemical moieties have been described previously (U.S. Pat. No. 6,333,149; WO 00/75364; Pellecchia et al., J. Biomol. NMR, 22:165-173 (2002)). For example, NMR methods can be used to determine an appropriate position on a specificity ligand for attaching chemical moieties so that the chemical moieties are directed to the conserved site when the specificity ligand is bound to the specificity site. Rather than determining a complete structure of the receptor-ligand complex, NMR can be used to obtain information on the proximity of atoms in a receptor-ligand complex, which in turn can be used to orient a ligand relative to the binding site of a receptor. The orientation of the ligand in a binding site allows the determination of a suitable position on the ligand to attach a chemical moiety for binding to a second site on the receptor.

To perform the NMR experiments, a target receptor is expressed in an organism such as bacteria, yeast, or other suitable organisms that can be grown on defined media. The organism can be grown in the presence of D₂O in place of water so that the receptor is deuterated. This is particularly useful when analyzing larger proteins. The organism can be grown in the presence of labels suitable for NMR analysis, for example, ¹⁵N, ³⁷C and the like. For example, a nitrogen or carbon source can be chosen to incorporate NMR label into amino acids of the target receptor, or the organism can be grown in the presence of labeled amino acids. Methods for isotopically labeling proteins are well known to those skilled in the art (Pellecchia et al., J. Biomol. NMR, 22:165-173 (2002); Pellecchia et al., J. Am. Chem. Soc. 123:4633-4634 (2001); Kay, Biochem. Cell Biol. 75:1-15 (1997); Laroche, et al., Biotechnology 12:1119-1124 (1994); LeMaster Methods Enzymol. 177:23-43 (1989); Muchmore et al., Methods Enzymol. 177:44-73 (1989); Reilly and Fairbrother, J. Biomolecular NMR 4:459-462 (1994); Ventors et al., J. Biomol. NMR 5:339-344 (1995); and Yamazaki et al., J. Am. Chem. Soc. 116:11655-11666 (1994), each of which is incorporated herein by reference). The use of NMR spectroscopy to identify amino acids involved in ligand interactions has been described previously (Davis et al., J. Biomolecular NMR 10:21-27 (1997); Hrovat et al., J. Biomolecular NMR 10:53-62 (1997); and Sem et al., J. Biol. Chem. 272:18038-18043 (1997), each of which is incorporated herein by reference).

In order to define which NMR cross peaks belong to amino acid residues in the part of a conserved site or specificity site that are proximal to each other, NMR experiments can be performed with the target receptor in the presence of a common ligand and/or specificity ligand that provides information on the orientation of the specificity site of the receptor relative to the conserved site (see U.S. Pat. No. 6,333,149; WO 00/75364). The proximity of receptor amino acid residues in a conserved site or specificity site to a bound ligand can be determined by nuclear Overhauser effect (NOE) experiments. Since an NOE is only observed between two protons that are within 5 Å of each other, NOE measurements between the receptor protons and a bound ligand indicate which protons on the ligand are within 5 Å of the protons on the receptor. Information on the interactions between receptor and ligand can be obtained using heteronuclear NMR experiments, including 2D HSQC, 3D HSQC-NOESY and 3D NOESY-HSQC (Cavanagh et al., in Protein NMR Spectroscopy: Principles and Practice, Academic Press, San Diego (1996), which is incorporated herein by reference).

Other methods can also be used to identify the position on a ligand to direct a moiety to a conserved site or specificity site of a receptor. For example, methods well know for structure-based drug design can be used to orient a common ligand and specificity ligand for simultaneous binding to a conserved site and specificity, respectively, of a receptor (see, for example, Gane and Dean, Curr. Opin. Struct. Biol. 10:401-404 (2000); Klebe, J. Mol. Med. 78:245-246 (2000); Kubinyi, Curr. Op. Drug Discov. Develop. 1:4-15 (1998); Muegge and Rarey, Reviews in Computational Chemistry, Volume 17, Lipkowitz and Boyd, eds., pp. 1-60, Wiley-VCH, New York (2001)). Other methods for identifying appropriate positions to orient a common ligand and specificity ligand to a conserved and specificity site, respectively, include methods based on biological mechanisms. For example, a position on a substrate analog having a structure similar to a natural substrate can be predicted to be proximal to a conserved site or specificity site based on the proximity of a group to a reactive group on a natural common ligand. As an example, in the case of NADH, the proton to be transferred from NADH to a substrate is required to be proximal to the specificity site since the proton must be transferred from NADH to the substrate. Thus, based on the biological activity and/or enzyme mechanism, a position on NADH, or a substantially similar substrate analog, can be predicted as one suitable for attaching a moiety for simultaneous binding of a conserved site and specificity site of a receptor.

In some cases, a common ligand and specificity ligand can be coupled to provide proper orientation for simultaneous binding to the conserved site and specificity site. However, if a common ligand and specificity ligand cannot be coupled to provide proper orientation, an expansion linker can be attached to facilitate simultaneous binding to the specificity site and conserved site of a receptor. Thus, the invention provides methods where a specificity ligand is attached to an expansion linker, and the expansion linker is further attached to a plurality of chemical moieties where the expansion linker has sufficient length and orientation to direct a specificity ligand to a specificity site and a plurality of chemical moieties to a conserved site of a receptor. Similarly, an expansion linker can be used to tether a common ligand to a plurality of chemical moieties for binding to a conserved site and specificity site, respectively. Exemplary expansion linkers are described in more detail below.

Once an expansion linker is synthetically attached to the specificity ligand, NMR experiments can be performed to establish that the modified specificity ligand contacts the same binding site atoms. In addition to NMR experiments, steady-state inhibition experiments can be performed to establish that adding the expansion linker does not significantly disrupt the strength of the binding interactions. Once a specificity ligand-expansion linker has been identified that binds to the specificity site in the correct orientation for attaching a common ligand to the expansion linker, a population of bi-ligands is generated, as described herein. The bi-ligands are generated by attaching potential common ligands having reactive groups to the expansion linker at the position on the expansion linker that orients the common ligand to the common ligand site. NMR methods for determining appropriate positions to attach a common ligand and specificity ligand to an expansion linker have been described previously (see U.S. Pat. No. 6,333,149 and WO 00/75364).

The methods of the invention can be used to identify a common ligand for a receptor family. A plurality of chemical moieties is attached to a specificity ligand and screened for binding to a receptor in a receptor family. The potential common ligands attached to a specificity ligand are screened for competitive binding, as described herein. Once a common ligand is identified, a plurality of chemical moieties can be attached to the common ligand and screened for binding to a specificity site. In this way, diversity can be increased in a common ligand, specificity ligand and/or expansion linker while maintaining the focus of a library on binding to a receptor family.

The plurality of chemical moieties that are potential common ligands, either common ligands or specificity ligands, can be a broad range of compounds of various structures generated by methods well known to those skilled in the art, as described below in more detail. Methods for producing pluralities of compounds to use as ligands, including common ligands or specificity ligands, are well known to those skilled in the art (Gordon et al., J. Med. Chem. 37: 1233-1251 (1994); Gordon et al., J. Med. Chem. 37: 1385-1401 (1994); Gordon et al., Acc. Chem. Res. 29:144-154 (1996); Wilson and Czarnik, eds., Combinatorial Chemistry: Synthesis and Application, John Wiley & Sons, New York (1997), each of which is incorporated herein by reference). A number of formats for generating combinatorial libraries are well known in the art, for example soluble libraries, compounds attached to resin beads, silica chips or other solid supports, including, for example, the “split resin approach” (U.S. Pat. No. 5,010,175; Gallop et al., J. Med. Chem., 37:1233-1251 (1994), each of which is incorporated herein by reference).

Compounds in a combinatorial library can be synthesized by the addition of one or more substituent groups to a base structure. Examples of substituent groups suitable for addition to a base structure include halo, hydroxy and protected hydroxyls, cyano, nitro, C₁ to C₆ alkyls, C₂ to C₇ alkenyls, C₂ to C₇ alkynyls, C₁ to C₆ substituted alkyls, C₂ to C₇ substituted alkenyls, C₂ to C₇ substituted alkynyls, C₁ to C₇ alkoxys, C₁ to C₇ acyloxys, C₁ to C₇ acyls, C₃ to C₇ cycloalkyls, C₃ to C₇ substituted cycloalkyls, C₅ to C₇ cycloalkenyls, C₅ to C₇ substituted cycloalkenyls, a heterocyclic ring, C₇ to C₁₂ phenylalkyls, C₇ to C₁₂ substituted phenylalkyls, phenyl and substituted phenyls, naphthyl and substituted naphthyls, cyclic C₂ to C₇ alkylenes, substituted cyclic C₂ to C₇ alkylenes, cyclic C₂ to C₇ heteroalkylenes, substituted cyclic C₂ to C₇ heteroalkylenes, carboxyl and protected carboxyls, hydroxymethyl and protected hydroxymethyls, amino and protected aminos, (monosubstituted)amino and protected (monosubstituted)aminos, (disubstituted)aminos, carboxamide and protected carboxamides, C₁ to C₄ alkylthios, C₁ to C₄ alkylsulfonyls, C₁ to C₄ alkylsulfoxides, phenylthio and substituted phenylthios, phenylsulfoxide and substituted phenylsulfoxides or phenylsulfonyl and substituted phenylsulfonyls. Substituent groups can also include compounds that are ligands to enzymes such as a specificity ligand or common ligand mimics, as well as expansion linkers.

In addition to synthesizing a plurality of chemical moieties, the plurality of chemical moieties can be a pool of commercially available molecules. The plurality of chemical moieties that are potential ligands can also be focused on compounds having structural similarities to a natural common ligand or a natural specificity ligand, or a mimic thereof. The pool of potential common ligands or specificity ligands can therefore be a group of variants, analogs and mimetics of a natural common ligand or natural specificity ligand, respectively. For example, the three-dimensional structure of a natural common ligand or a natural specificity ligand can be used to search commercially available databases of commercially available molecules such as the Available Chemicals Directory (MDL Information Systems, Inc.; San Leandro Calif.) and ASINEX (Moscow Russia) to identify potential common ligands having similar shape, electrochemical and/or physicochemical properties of a natural common ligand or natural specificity ligand. Methods for identifying molecules having similar structure are well known in the art and are commercially available (Doucet and Weber, in Computer-Aided Molecular Design: Theory and Applications, Academic Press, San Diego Calif. (1996); software is available from Molecular Simulations, Inc., San Diego Calif.; Chau and Dean, J. Mol. Graph. 5:97-100 (1989); Bladon, J. Mol. Graph. 7:130-137 (1989), THREEDOM software package, each of which is incorporated by reference). Furthermore, if structural information is available for the conserved site or specificity site in the receptor, particularly with a known ligand bound, compounds that fit the conserved site or specificity site can be identified through computational methods (Blundell, Nature 384 Supp:23-26 (1996), which is incorporated herein by reference).

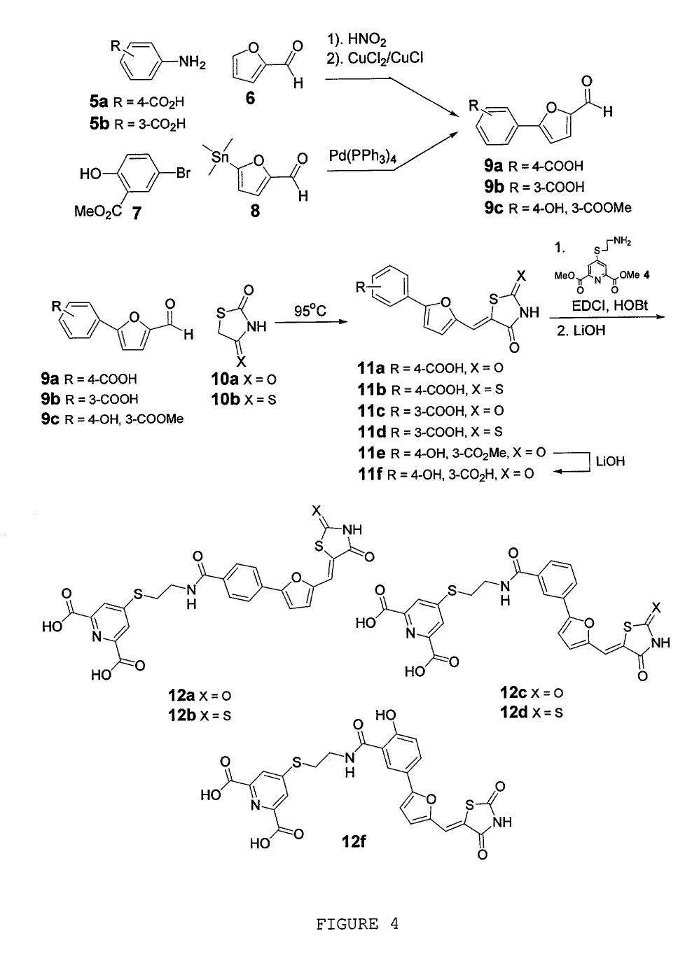

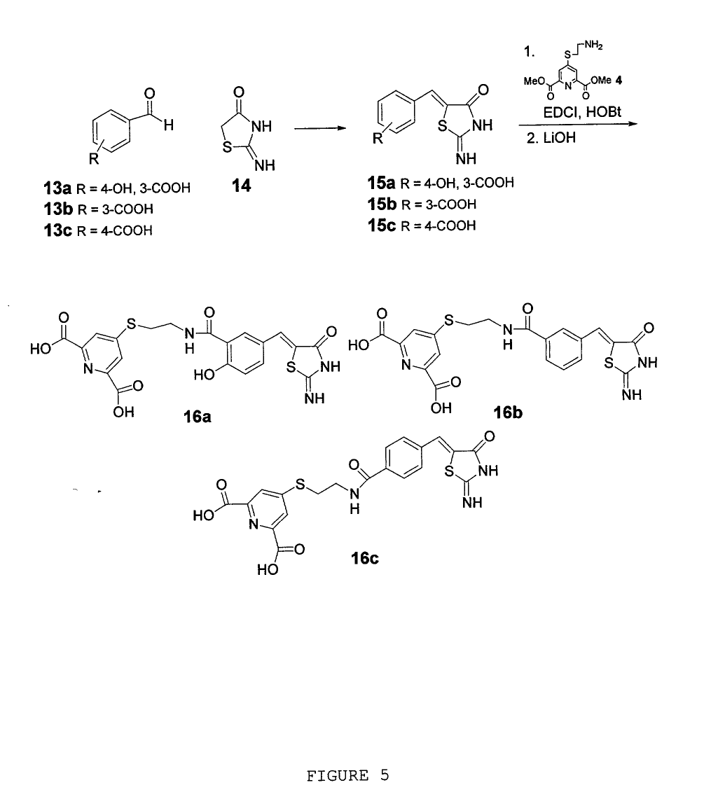

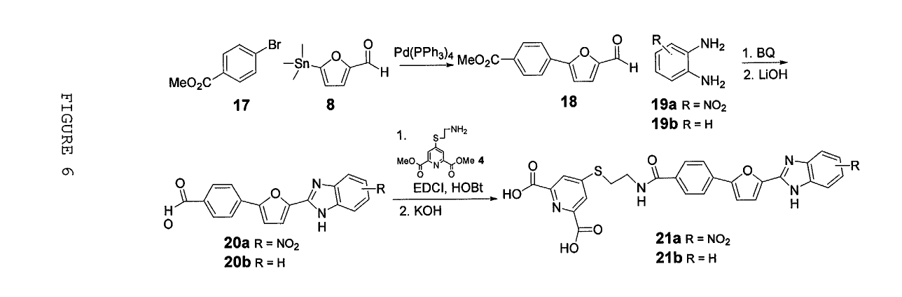

Exemplary methods for synthesizing a plurality of chemical moieties as potential common ligands are disclosed herein (see Examples II-IV). For example, methods are described for the synthesis of rhodanine and thiazolidinedione-based bi-ligand inhibitors (Example II and FIG. 4), pseudothiohydantoin-based bi-ligand inhibitors (Example III and FIG. 5), and benzimidazole-based bi-ligand inhibitors (Example IV and FIG. 6).

To facilitate orienting the specificity ligand and common ligand to a specificity site and conserved site, respectively, the specificity ligand and common ligand can be tethered by an expansion linker having sufficient length and orientation to direct the specificity ligand to the specificity site of a receptor and the common ligand to the conserved site of a receptor. The use of various expansion linkers can be used to increase diversity of a library. For example, a particular length of expansion linker can provide proper orientation in a subfamily of receptors but work less well for another subfamily, whereas a different linker can function better for other subfamilies. Thus, a library built with a common ligand library and a particular specificity ligand or a specificity ligand library and a particular common ligand can be further diversified by combining with various linkers.

A specificity ligand can be attached to a common ligand by an expansion linker, which is attached to the common ligand at a position so that the expansion linker is oriented towards the specificity site. An expansion linker has sufficient length and orientation to direct a specificity ligand to a specificity site. The expansion linker is designed to have at least two positions for attaching at least two ligands. One of the positions is used to attach the expansion linker to a common ligand. The other position is used for attaching a specificity ligand.

For some biligands, the expansion linker can be any molecule that provides sufficient length and orientation for directing a common ligand to a conserved site of a receptor. Therefore, any chemical group that provides the appropriate orientation and positioning of the common ligand relative to the specificity ligand for optimized binding to their respective sites on the receptor can be used as an expansion linker.

Expansion linkers that are useful for generating bi-ligands include, for example, substituted phosgene, urea, furane and salicylic acid, substituted piperidine, pyrrolidine, morpholine, 2,4 di-bromobenzoate, 2-hydroxy-1,4-naphthoquinone, tartaric acid, indole, isoindazole, 1,4-benzisoxazine, phenanthrene, carbazole, purine, pyrazole and 1,2,4-triazole.

As used herein, the term “linker” refers to a chemical group that can be attached to either the common ligand or the specificity ligand of a bi-ligand. The linker provides functional groups through which a common ligand and a specificity ligand are indirectly but covalently bound to one another. The linker can be a simple functional group, such as COOH, NH₂, OH, or the like. Alternatively, the linker can be a complex chemical group containing one or more unsaturation, one or more substituent, and/or one or more heterocyclic atom.

A linker can be, for example, an alkyl group. As used herein, “alkyl” means a carbon chain having from one to twenty carbon atoms. The alkyl group of the present invention can be straight chain or branched. It can be unsubstituted or can be substituted. When substituted, the alkyl group can have up to ten substituent groups, such as COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN or X where R₉, R₁₀, and R₁₁ each independently are hydrogen, alkyl, alkenyl, alkynyl, aryl, or heterocycle, or R₉ and R₁₀ together with the nitrogen atom to which they are attached can be joined to form a heterocyclic ring.

Additionally, the alkyl group present in the compounds of the invention, whether substituted or unsubstituted, can have one or more of its carbon atoms replaced by a heterocyclic atom, such as an oxygen, nitrogen, or sulfur atom. For example, alkyl as used herein includes groups such as (OCH₂CH₂)n or (OCH₂CH₂ CH₂)n, where n has a value such that there are twenty or less carbon atoms in the alkyl group. Similar compounds having alkyl groups containing a nitrogen or sulfur atom are also encompassed by the present invention.

A linker can also be an alkenyl group. As used herein “alkenyl” means an unsaturated alkyl groups as defined above, where the unsaturation is in the form of a double bond. The alkenyl groups of the present invention can have one or more unsaturations. Nonlimiting examples of such groups include CH═CH₂, CH₂CH₂CH═CHCH₂CH₃, and CH₂CH═CHCH₃. As used herein “alkynyl” means an unsaturated alkyl group as defined above, where the unsaturation is in the form of a triple bond. Alkynyl groups of the present invention can include one or more unsaturations. Nonlimiting examples of such groups include C≡CH, CH2CH2C≡CCH₂CH₃, and CH₂C≡CCH₃.

The linkers can include compounds in which R₁ to R₆ each independently are complex substituents containing one or more unsaturation, one or more substituent, and/or one or more heterocyclic atom. These complex substituents are also referred to herein as “linkers” or “expansion linkers.”

A linker can additionally be a heterocyclic group. As used herein, “heterocyclic group” or “heterocycle” refers to an aromatic compound or group containing one or more heterocyclic atom. Nonlimiting examples of heterocyclic atoms that can be present in the heterocyclic groups include nitrogen, oxygen and sulfur. In general, heterocycles of the present invention will have from five to seven atoms and can be substituted or unsubstituted. When substituted, substituents include, for example, those groups provided for R₁ to R₁₀. Nonlimiting examples of heterocyclic groups of the invention include pyroles, pyrazoles, imidazoles, pyridines, pyrimidines, pyridzaines, pyrazines, triazines, furans, oxazoles, thiazoles, thiophenes, diazoles, triazoles, tetrazoles, oxadiazoles, thiodiazoles, and fused heterocyclic rings, for example, indoles, benzofurans, benzothiophenes, benzoimidazoles, benzodiazoles, benzotriazoles, and quinolines. When compounds of the invention contain a linker, the linker can be present, for example, at any position on a ligand compounds.

Another group of expansion linkers includes molecules containing phosphorous. These phosphorus-containing molecules include, for example, substituted phosphate esters, phosphonates, phosphoramidates and phosphorothioates. The chemistry of substitution of phosphates is well known to those skilled in the art (Emsley and Hall, The Chemistry of Phosphorous: Environmental, Organic, Inorganic and Spectroscopic Aspects, Harper & Row, New York (1976); Buchwald et al., Methods Enzymol. 87:279-301 (1982); Frey et al., Methods Enzymol. 87:213-235 (1982); Khan and Kirby, J. Chem. Soc. B:1172-1182 (1970), each of which is incorporated herein by reference). A related category of expansion linkers includes phosphinic acids, phosphonamidates and phosphonates, which can function as transition state analogs for cleavage of peptide bonds and esters as described previously (Alexander et al., J. Am. Chem. Soc. 112:933-937 (1990), which is incorporated herein by reference). The phosphorous-containing molecules useful as expansion linkers can have various oxidation states, both higher and lower, which have been well characterized by NMR spectroscopy (Mark et al., Progress in NMR Spectroscopy 16:227-489 (1983), which is incorporated herein by reference). However, any reactive chemical group that can be used to position a common ligand and a specificity ligand in an optimized position for binding to their respective sites can be used as an expansion linker.

Reactive groups on an expansion linker and the ligands to be attached to the expansion linker should be reactive so as to generate a covalent attachment of the common ligand or specificity ligand to the expansion linker in the orientation for binding to their respective binding sites on the receptor. A particularly useful reaction is that of a nucleophile reacting with an electrophile. Thus, the expansion linker and ligands can have reactive groups suitable for coupling the expansion linker to a ligand, and the placement of a nucleophile or electrophile on an expansion linker or ligand can be chosen based on the needs of the synthetic schemes used to generate the compounds.

Other suitable reactive groups include carbon-carbon bond forming reactions or other bond formation via catalysts. Reactions suitable for carbon-carbon bond formation include reactions such as Heck reactions, Suzuki reactions, Stille reactions, and the like (Tsuji, Palladium Reagents and Catalysts, J., John Wiley and Sons Ltd (1997); Hassner and Stumer, Organic Syntheses Based on Name Reactions and Unnamed Reactions Pergamon (1994). Additional suitable reactions include olefin metathesis (Furstner, Alkene Metathesis in Organic Synthesis; Springer, Berlin (1998); Armstrong, S. K. J. Chem. Soc., Perkin Trans. 1:371-388 (1998); Grubbs and Chang, Tetrahedron 54:4413-4450 (1998); Wright, Curr. Org. Chem. 3:211-240 (1999); Hoveyda and Schrock, Chem. Eur. J. 7:945-950 (2001)).

It is understood that any suitable reactive groups, including those described herein, can be used to couple a specificity ligand and common ligand directly or via an expansion linker so that the common ligand and specificity ligand are oriented for binding to a conserved site and specificity site, respectively. Reactive chemical groups for coupling chemical compounds are well known to those skilled in the art (see, for example, Parlow et al., J. Org. Chem. 62:5908 (1997); Flyn et al., Med. Chem. Res. 8:219 (1998), each of which is incorporated herein by reference).

Many of the expansion linkers exemplified above have electrophilic groups available for attaching ligands. Electrophilic groups useful for attaching ligands include electrophiles such as carbonyls, alkenes, activated esters, acids and alkyl and aryl halides. The expansion linkers having electrophilic groups can be attached to ligands having nucleophilic groups positioned for attachment of the ligands in an orientation for binding of the common ligand and specificity ligand to a conserved site and specificity site, respectively. Desirable common ligands can have, for example, alcohols, amines, or mercaptans. However, if a common ligand is identified that does not have appropriate reactive groups for attaching a ligand in a desired orientation to the expansion linker or if the ligand cannot be modified to generate an appropriate reactive group in a desired position, an additional screen can be performed, as described above, to identify a common ligand having desired binding characteristics as well as a chemical group in the proper position to achieve a desired orientation of ligands after covalently linking a ligand to the expansion linker.

Reactive positions on the expansion linker can be modified, for example, with hydroxyl, amino or mercapto groups. Therefore, ligands containing reactive hydroxyl, amino or mercapto groups positioned so that, after attaching a specificity ligand, the expansion linker orients the common ligand and specificity ligand to their respective sites on the receptor can be reacted with the expansion linkers described above.

Once a population of molecules containing a specificity ligand and a plurality of chemical moieties is generated, either attached directly or via an expansion linker, the population is screened for binding activity to a receptor that binds the specificity ligand. Furthermore, a population of bi-ligands having diversity at the specificity site can be screened for binding activity to a receptor in a corresponding receptor family. Methods for screening for binding activity are well known to those skilled in the art. For example, competitive binding assays using a detectable ligand can be used to screen for binding activity of a ligand (see Examples VII-XI).

Methods of screening for binding of a common ligand, specificity ligand, or bi-ligand are well known in the art. For example, a receptor can be incubated in the presence of a known ligand and one or more potential ligands, either a common ligand, specificity ligand or bi-ligand. In some cases, a natural common ligand or specificity ligand has an intrinsic property that is useful for detecting whether the natural ligand is bound. For example, the natural common ligand for dehydrogenases, NAD, has intrinsic fluorescence. Therefore, increased fluorescence in the presence of potential common ligands due to displacement of NAD can be used to detect competition for binding of NAD to a target NAD binding receptor (Li and Lin, Eur. J. Biochem. 235:180-186 (1996); and Ambroziak and Pietruszko, Biochemistry 28:5367-5373 (1989), each of which is incorporated herein by reference).

In other cases, when a natural ligand does not have an intrinsic property useful for detecting ligand binding, the known ligand can be labeled with a detectable moiety, for example, a fluor, radioisotope, chromogen, and the like. For example, the natural common ligand for kinases, ATP, can be radiolabeled with ³²P, and the displacement of radioactive ATP from an ATP binding receptor in the presence of potential common ligands can be used to detect additional common ligands. Any detectable moiety, for example, a radioactive, fluorescent or calorimetric label, can be added to the known ligand so long as the labeled known ligand can bind to a receptor having a conserved site. Such detectable moieties can also be coupled to a specificity ligand if the desired assay is to measure competition of a specificity ligand.

When screening for a common ligand from a plurality of chemical moieties attached to a specificity ligand, the common ligand is generally identified by exhibiting increased binding affinity relative to the specificity ligand alone, that is, the parent specificity ligand. Similar increases in binding affinity of a specificity ligand screened from a plurality of chemical moieties by comparison to the common ligand alone, that is, the parent common ligand, can also be used to identify a specificity ligand. Such an increase in binding affinity is measurable by the assay used and is at least 2-fold higher affinity than the parent common or specificity ligand alone, for example, at least 3-fold higher, 4-fold higher, 5-fold higher, 7-fold higher, 10-fold higher, 20-fold higher, 50-fold higher, 100-fold higher, 200-fold higher, 300-fold higher, 500-fold higher, 1000-fold higher, 10,000-fold higher or even greater. As discussed above, due to the linking of two ligands that bind to independent sites on a receptor, the increase in binding affinity can be synergistic, with increases in binding affinity by orders of magnitude.

The invention also provides a method of generating a population of bi-ligands to a receptor in a receptor family. The method includes the steps of coupling a common ligand identified by the methods disclosed herein to a plurality of chemical moieties at a position on the common ligand to direct the common ligand to a conserved site and the plurality of chemical moieties to a specificity site of a receptor in a receptor family.

The methods of the invention are useful for generating populations or libraries of bi-ligands that are suitable for screening and identifying bi-ligands having specificity for particular members of a receptor family. Because the population or library of bi-ligands contains a common ligand, which can bind to multiple members of a receptor family, the same population or library can be used repeatedly to screen for bi-ligands specific to various members of the same receptor family.

The invention also provides a method of identifying a bi-ligand to a receptor in a receptor family. The method can include the steps of generating a population of molecules comprising a common ligand identified by a method of the invention, the common ligand attached to a plurality of chemical moieties at a position on the common ligand to direct the common ligand to a conserved site and the plurality of chemical moieties to a specificity site of a receptor in a receptor family; screening the population of molecules for binding to a receptor in the receptor family; and identifying a bi-ligand having binding activity and specificity for the receptor. The receptor for which bi-ligands are identified can be the same as the receptor used for the initial screen for the common ligand or can be another receptor in the same receptor family. Such a method can further include repeating the steps one or more times to identify bi-ligands for other members of the receptor family.

Additionally provided is a method of identifying a population of bi-ligands to receptors in a receptor family. The method can include the steps of (a) generating a first population of molecules comprising a specificity ligand having binding activity for a receptor in a receptor family, the specificity ligand attached to a first plurality of chemical moieties at a position on the specificity ligand to direct the specificity ligand to a specificity site and the chemical moieties to a conserved site of the receptor; (b) screening the population of molecules for binding to the receptor; (c) identifying a bi-ligand having increased binding activity for the receptor relative to the specificity ligand alone, thereby identifying a common ligand having binding activity for the receptor; (d) generating a second population of molecules, the population comprising the common ligand identified in step (c) attached to a second plurality of chemical moieties at a position on the common ligand to direct the common ligand to a conserved site and the plurality of chemical moieties to a specificity site of a receptor in a receptor family; (e) screening the population of molecules for binding to a receptor in the receptor family; (f) identifying a bi-ligand having binding activity and specificity for the receptor; and (g) optionally repeating steps (e) and (f) one or more times for another receptor in the receptor family. Thus, the methods of the invention can be used to identify a bi-ligand having specificity for a receptor in a receptor family or the steps of the method optionally can be repeated to identify a population of bi-ligands, which can contain bi-ligands have specificity for at least one receptor in a receptor family or individual bi-ligands independently having specificity for various members of the receptor family.

When a common ligand is identified by screening a population of bi-ligands having a specificity ligand and a plurality of potential common ligands, the identified common ligand can be subsequently used to build a population of bi-ligands containing the newly identified common ligand. Such a population of bi-ligands can be screened for binding to the same receptor used to initially identify the common ligand, that is the receptor to which the specificity ligand binds. Furthermore, the population can be used to screen for binding to other members of the receptor family. Thus, the population can be used to screen for a bi-ligand to a receptor in a receptor family, either the original receptor used to identify the common ligand or another receptor. The population can further be screened to identify a bi-ligand specific for 2 or more receptors, 3 or more receptors, 4 or more receptors, 5 or more receptors, 6 or more receptors, 7 or more receptors, 8 or more receptors, 9 or more receptors, 10 or more receptors, 15 or more receptors, 20 or more receptors, or even a greater number of receptors in a receptor family. The methods of the invention using a common ligand and a plurality of chemical moieties as potential specificity ligands allows the same population to be used repeatedly to identify bi-ligands for multiple members of a receptor family.

Various methods can be used to determine whether a receptor is in a receptor family. Such methods can be used to confirm that an uncharacterized or newly identified gene encodes a member of a particular receptor family. Methods for determining that two receptors are in the same family, and thus constitute a receptor family, are well known in the art, as disclosed herein. Once a receptor has been identified as a member of a receptor family using computational methods, the receptor can be confirmed as a member of the receptor, for example, using competitive binding assays with a common ligand, as described above. Thus, receptors identified as members of a receptor family based on containing amino acid motifs and that bind to the same common ligand are considered to be members of the same receptor family.

Various algorithms can also be used to determine if a receptor is in a receptor family or if two receptors are in the same receptor family if the sequence of a receptor is known or has been newly identified. In such a case, the entire sequence of the members of the receptor family need not be known, only sufficient sequence information to determine that the receptors are in the same receptor family. One such computational method is BLAST, Basic Local Alignment Search Tool, which uses a heuristic algorithm that seeks local alignments and is therefore able to detect relationships among sequences which share only isolated regions of similarity (Altschul et al., J. Mol. Biol. 215:403-410 (1990), which is incorporated herein by reference; www.ncbi.nlm.gov/BLAST/).

Modifications of the BLAST algorithm can also be used (Altschul et al., Nucleic Acids Res. 25:3389-3402 (1997), which is incorporated herein by reference). One modification is Gapped BLAST, which allows gaps, either insertions or deletions, to be introduced into alignments. Allowing gaps in alignments tends to reflect biologic relationships more closely. A second modification is PSI-BLAST, which is a sensitive way to search for sequence homologs. PSI-BLAST performs an initial Gapped BLAST search and uses information from any significant alignments to construct a position-specific score matrix, which replaces the query sequence for the next round of database searching. A PSI-BLAST search is often more sensitive to weak but biologically relevant sequence similarities.

Another method for identifying members of a receptor family is PROSITE, which determines the function of uncharacterized proteins translated from genomic or cDNA sequences (Bairoch et al., Nucleic Acids Res. 25:217-221 (1997), which is incorporated herein by reference; us.expasy.org/prosite). PROSITE consists of a database of biologically significant sites and patterns that can be used to determine whether a sequence belongs to a known family of proteins. Functionally related proteins can be identified by the occurrence of a particular cluster of amino acid residues, which can be adjacent amino acids or have intervening amino acids and can be called a pattern, motif, signature or fingerprint. PROSITE uses a computer algorithm to search for motifs that identify proteins as family members. PROSITE also maintains a compilation of previously identified motifs, which can be used to determine if a newly identified protein is a member of a known protein family.

Still another method for identifying members of a receptor family is Structural Classification of Proteins (SCOP). Similar to PROSITE, SCOP maintains a compilation of previously determined protein motifs for comparison and determination of related proteins (Murzin et al., J. Mol. Biol. 247:536-540 (1995), which is incorporated herein by reference; scop.mrc-lmb.cam.ac.uk/scop/).

Other methods useful for determining whether a receptor is in a receptor family or whether two receptors are in the same receptor family are well known to those skilled in the art, for example, Attwood et al., Nucl. Acids. Res. 30:239-241 (2002); Apweiler et al. Nucl. Acids Res. 29:37-40 (2001); Hofmann et al., Nucl. Acids Res. 27:215-219 (1999); Bucher and Bairoch, in Proceedings 2nd International Conference on Intelligent Systems for Molecular Biology, Altman et al., eds., pp. 53-61, AAAI Press, Menlo Park (1994); Henikoff et al., Nucl. Acids Res. 28:228-230 (2000); Henikoff et al., Bioinformatics 15:471-479 (1999); Henikoff and Henikoff, Genomics 19:97-107 (1994); Henikoff et al., Gene 163:GC17-26 (1995); Pietrokovski et al., Nucl. Acids Res. 24:3836-3845 (1996); Rose et al., Nucl. Acids Res. 26:1628-1635 (1998); Corpet et al., Nucl. Acids Res. 28:267-269 (2000); Worley et al., Bioinformatics 14:890-891 (1998); Smith et al., Genome Res. 6:454-462 (1996); Worley et al., Genome Res. 5:173-184 (1995); Sonnhammer et al., Proteins: Structure Function Genet. 28:405-420 (1997); Sonnhammer et al., Nucl. Acids Res. 26:320-322 (1998); Bateman et al., Nucl. Acids Res. 27:260-262 (1999); Bateman et al., Nucl. Acids Res. 28:263-266 (2000); Bateman et al., Nucl. Acids Res. 30:276-280 (2002); and Durbin et al., Biological Sequence Analysis: Probabilistic Models of Proteins and Nucleic Acids, Cambridge University Press (1998), each of which is incorporated herein by reference.

Exemplary resources for determining whether a receptor is in a receptor family and for identifying motifs of a receptor family include, for example:

PROSITE (us.expasy.org/prosite);

BLOCKS (www.blocks.fhcrc.org);

PRINTS (bioinf.man.ac.uk/ddbrowser/PRINTS);

BCM Search Launcher (searchlauncher.bcm.tmc.edu);

PRODOM (prodes.toulouse.inra.fr/prodom/doc/prodom.html);

PATSCAN (www-unix.mcs.anl.gov/compgio/PatScan//HTML/patscan.html);

PATTERNFIND (www.isrec.isb-sib.ch/software/PATFND_form.html);

PMOTIF (alces.med.umn.edu/dbmotif.html);

HMMER (hmmr.wustl.edu);

BLAST (www.ncbi.nlm.nih.gov/BLAST);

BLITZ (www2.ebi.ac.uk);

FASTA (www.ebi.ac.uk/fasta33);

SCOP (scop.mrc-lmb.cam.ac.uk/scop);

PFAM (pfam.wustl.edu);

PIX (www.hgmp.mrc.ac.uk/Registered/Webapp/pix); and

INTERPRO (www.ebi.ac.uk/interpro).

Examplary functional amino acid motifs include the Rossman fold, which includes GXXGXXG (SEQ ID NO:1) or GXGXXG (SEQ ID NO:2) and is present in enzymes that bind nucleotides (Brandon and Tooze, in Introduction to Protein Structure, Garland Publishing, New York (1991); Creighton, Proteins: Structures and Molecular Principles, p.368, W. H. Freeman, New York (1984); Rossman et al., in The Enzymes Vol 11, Part A, 3rd ed., Boyer, ed., pp. 61-102, Academic Press, New York (1975); Wierenga et al., J. Mol. Biol. 187:101-107 (1986); and Ballamacina, FASEB J. 10:1257-1269 (1996), each of which is incorporated herein by reference). Other exemplary amino acid motifs include GXGGXXXG (SEQ ID NO: 3), a second motif is KXEX₆SXKX₅₋₆M (SEQ ID NO: 4), and a third motif is PXNPTG (SEQ ID NO: 5), which are found in pyridoxal binding receptors (Suyama et al., Protein Engineering 8:1075-1080 (1995), which is incorporated herein by reference).

After a receptor family has been selected for development of bi-ligands as drug candidates, a member of the receptor family is selected for identification of a common ligand and a common ligand is identified, as described above. For developing bi-ligands having specificity for a member of a receptor family, at least two receptors in the receptor family are selected as drug targets for identifying ligands useful as therapeutic agents. The criteria for selection of receptor family members depend on the needs of the user.

Since the selected receptor family members will be screened for binding to common ligands and/or bi-ligands, the selected members are produced or isolated in quantities suitable for performing desired screening assays. For example, after a target receptor is selected, the selected receptor(s) can be cloned and expressed using well known methods of gene cloning and expression. The target receptor gene can be cloned into an appropriate expression vector for expression in bacteria, insect cells, yeast cells, mammalian cells, and the like. Methods of gene cloning and expression are well known to those skilled in the art (Sambrook et al., Molecular Cloning: A Laboratory Manual, 2nd ed., Cold Spring Harbor Press, Plainview, N.Y. (1989); Ausubel et al., Current Protocols in Molecular Biology (Supplement 56), John Wiley & Sons, New York (2001); Sambrook and Russel, Molecular Cloning: A Laboratory Manual, 3rd ed., Cold Spring Harbor Press, Cold Spring Harbor (2001); Dieffenbach and Dveksler, eds., PCR Primer: A Laboratory Manual, Cold Spring Harbor Laboratory Press, Plainview, N.Y. (1995)).

Various criteria can be used to select a member of a receptor family depending on whether the receptor is used to identify a common ligand or to generate a population of bi-ligands suitable for screening drug candidates. For identifying a common ligand using methods of the invention, a target receptor family member can be selected based on the convenience of expressing the target receptor, known characteristics of the receptor, such as whether there is a known common ligand or specificity ligand, and the like.

For developing therapeutic agents, the receptor family can include members from a pathogenic organism, and the receptor family members selected can be those most divergent from the organism to be treated with the therapeutic agent. For example, if the organism to be treated is a mammal such as human, then the receptor family members from the pathogenic organism can be compared to known mammalian or human members of the receptor family. Methods of comparing protein sequences are well known in the art and include BLAST and other methods disclosed herein. A receptor in the target pathogenic organism can be chosen as one distantly related to human for identifying ligands as therapeutic agents since it is easier to identify ligands having higher specificity for the pathogenic organism if the target receptor in the pathogenic organism is divergent from the organism being treated.

If the receptor family is from a target cell such as a cancer cell, target receptors in a receptor family can be selected based on the criteria that the target receptor is more highly expressed or is more active in a cancer cell. A ligand targeted to such a receptor will be more likely to affect the target cancer cell rather than other non-cancerous cells in the organism.

The methods of the invention can be used to develop candidate drugs, for example, enzyme inhibitors, suitable for treating a target disease by selecting a target receptor associated with the disease. Selection of a target receptor associated with a target disease allows the development of a ligand useful as a therapeutic agent for the target disease. After identification of a target disease, a cell or organism responsible for the target disease is selected, and a receptor family expressed in the organism is identified for targeting of a ligand. For example, a pathogen can be selected as the target organism to develop drugs effective in combating a disease caused by that pathogen. Any pathogen can be selected as a target organism. Examples of pathogens include, for example, viruses, bacteria, fungi or protozoa. In addition, a target cell such as a cancer cell can be selected to identify drugs effective for treating cancer. Examples of such target cells include, for example, breast cancer, prostate cancer, and ovarian cancer cells as well as leukemia, lymphomas, melanomas, sarcomas and gliomas.

Exemplary pathogenic bacteria, which can be selected as target organisms, include Staphylococcus, Mycobacteria, Mycoplasma, Streptococcus, Haemophilus, Neisseria, Bacillus, Clostridium, Corynebacteria, Salmonella, Shigella, Vibrio, Campylobacter, Helicobacter, Pseudomonas, Legionella, Bordetella, Bacteriodes, Fusobacterium, Yersinia, Actinomyces, Brucella, Borrelia, Rickettsia, Ehrlichia, Coxiella, Chlamydia, and Treponema. Pathogenic strains of Escherichia coli can also be target organisms.

Ligands targeted to receptors in these pathogenic bacteria are useful for treating a variety of diseases including bacteremia, sepsis, nosocomial infections, pneumonia, pharyngitis, scarlet fever, necrotizing fasciitis, abscesses, cellulitis, rheumatic fever, endocarditis, toxic shock syndrome, osteomyelitis, tuberculosis, leprosy, meningitis, pertussis, food poisoning, enteritis, enterocolitis, diarrhea, gastroenteritis, shigellosis, dysentery, botulism, tetanus, anthrax, diphtheria, typhoid fever, cholera, actinomycosis, Legionnaire's disease, gangrene, brucellosis, lyme disease, typhus, spotted fever, Q fever, urethritis, vaginitis, gonorrhea and syphilis.

Exemplary target organisms selected from yeast and fungi include pathogenic yeast and fungi such as Aspergillus, Mucor, Rhizopus, Candida, Cryptococcus, Blastomyces, Coccidioides, Histoplasma, Paracoccidioides, Sporothrix, and Pneumocystis. Ligands targeted to receptors in these pathogenic yeast and fungi are useful for treating a variety of diseases including aspergillosis, zygomycosis, candidiasis, cryptococcoses, blastomycosis, coccidioidomycosis, histoplasmosis, paracoccidioidomycosis, sporotrichosis, and pneuomocystis pneumonia.

Exemplary target organisms selected from protozoa include Plasmodium, Trypanosoma, Leishmania, Toxoplasma, Cryptosporidium, Giardia, and Entamoeba. Ligands targeted to receptors in these pathogenic protozoa are useful for treating a variety of diseases including malaria, sleeping sickness, Chagas' disease, leishmaniasis, toxoplasmosis, cryptosporidiosis, giardiasis, and amebiasis.

The target receptor can be further validated as a useful therapeutic target by determining if the selected target receptor is known to be required for normal growth, viability or infectivity of the target organism or cell. Such methods can include, for example, gene knockout experiments to test the function of a target receptor (Ausubel et al., Current Protocols in Molecular Biology, Vols 1-3, John Wiley & Sons (1998); (Benson and Goldman, J. Bacteriol. 174:167301681 (1992); Hughes and Roth, Genetics 119:9-12 (1988); and Elliot and Roth, Mol. Gen. Genet. 213:332-338 (1988); Wang, Parasitology 114:531-544 (1997); and Li et al, Mol. Biochem. Parasitol. 78:227-236 (1996), each of which is incorporated herein by reference).

The methods disclosed herein allow the identification of a common ligand for a receptor family. As disclosed herein, variants of a parent common ligand can be generated and screened for optimized binding activity. Thus, in addition to identifying common ligands, the methods can also be used to optimize a common ligand.

The invention additionally provides a method for identifying an optimized common ligand. The method can include the steps of generating a population of bi-ligands, the population comprising a specificity ligand having binding activity for a receptor in a receptor family, the specificity ligand attached to an expansion linker, the expansion linker further attached to a plurality of common ligand variants, the expansion linker having sufficient length and orientation to direct the specificity ligand to a specificity site and the common ligand to a conserved site of the receptor; screening the population of bi-ligands for optimized binding of a bi-ligand to the receptor, thereby identifying an optimized common ligand; and isolating the optimized common ligand.

The method of identifying an optimized common ligand can further include the steps of attaching an expansion linker to the optimized common ligand, wherein the expansion linker has sufficient length and orientation to direct a second ligand to a specificity site of a receptor in the receptor family, to form an optimized common ligand module; and generating a population of bi-ligands, the population comprising the optimized common ligand module attached to a plurality of variable chemical moieties. The method can additionally include the steps of screening the population of bi-ligands comprising the optimized common ligand module for binding to a first receptor in the receptor family; and identifying a bi-ligand having binding activity for the first receptor.

Additionally, the invention provides a method for generating a population of bi-ligands. The method can include the steps of generating a first population of bi-ligands, the population comprising a specificity ligand having binding activity for a receptor in a receptor family, the specificity ligand attached to an expansion linker, the expansion linker further attached to a plurality of common ligand variants, the expansion linker having sufficient length and orientation to direct the specificity ligand to a specificity site and the common ligand variants to a conserved site of the receptor; screening the population of bi-ligands for optimized binding of a bi-ligand to the receptor, thereby identifying an optimized common ligand; generating an optimized common ligand module, the module comprising the identified optimized common ligand attached to an expansion linker having sufficient length and orientation to direct a second ligand to a specificity site of a receptor in the receptor family; and generating a second population of bi-ligands, the population comprising the optimized common ligand module attached to a plurality of variable chemical moieties. The method of generating a population of bi-ligands having an optimized common ligand can further include the steps of screening the population of bi-ligands comprising the optimized common ligand module for binding to a first receptor in the receptor family; and identifying a bi-ligand having binding activity for the first receptor. It is understood that an optimized common ligand can be directly attached to a specificity ligand or can be tethered via an expansion linker, as described above.

As used herein, a “common ligand variant” refers to a derivative of a common ligand. A common ligand variant has structural similarities to a parent common ligand. A common ligand variant differs from another variant, including the parent common ligand, by at least one atom. For example, as with NAD and NADH, the reduced and oxidized forms differ by an atom and are therefore considered to be variants of each other.

As used herein, the term “optimized common ligand” refers to a common ligand variant having improved binding characteristic relative to a parent common ligand. A parent common ligand can be a natural common ligand or a common ligand identified by the methods described herein. An improved binding characteristic can be, for example, increased binding affinity, increased specificity, improved biological activity, and/or an improved pharmacological property such as improved absorption, distribution, metabolism and/or elimination (ADME). In addition, an improved binding characteristic can be essentially the same binding activity as a parent common ligand. In such a case, an optimal binding ligand having substantially similar activity can provide an alternative common ligand structure onto which additional variability can be introduced, as in a bi-ligand.

The population of bi-ligands having common ligand variants is screened for optimized activity of a bi-ligand. Optimized activity can be, for example, increased binding affinity, or other improved binding characteristics, as described above. One skilled in the art can readily determine appropriate assays to identify an improved activity, either using in vitro or in vivo assays, depending on the activity being measured. Methods for measuring biological and/or pharmacological activity of a compound are well known to those skilled in the art (Estes, Mayo Clin. Proc. 73:1114-1122 (1998); Zak and Sande, Handbook of Animal Models of Infection Academic Press San Diego, Calif. (1999).

In addition, optimized binding can be, for example, essentially equivalent binding to a parent common ligand from which the common ligand variants are derived. In such a case, optimized binding is provided by the modification of a common ligand that still allows binding activity. An optimized common ligand having essentially the same activity as a parent common ligand can be used to increase the diversity of a population of bi-ligands suitable for screening for binding activity to a receptor family. Even relative minor modifications to a common ligand that alone do not significantly increase binding activity can provide diversity that, when combined with variable potential specificity ligands, increases the number of bi-ligands that can potentially bind to a greater number of receptors in a receptor family. Thus, depending on whether the desire is to increase binding affinity or increase diversity, one skilled in the art can recognize whether a bi-ligand in a population of bi-ligands containing common ligand variants has optimized binding activity.

The screening and identification of a bi-ligand exhibiting optimized binding to a receptor provides the identification of the corresponding common ligand variant as an optimized common ligand. Once an optimized common ligand is identified, the optimized common ligand can be isolated. The isolation of the optimized common ligand can be performed by cleaving the optimized common ligand from the identified bi-ligand exhibiting optimized binding to the receptor used for screening. Alternatively, the optimized common ligand can be synthesized de novo, resulting in an isolated common ligand. Following the cleavage or synthesis, the isolated optimized common ligand can include an expansion linker, if desired. If the optimized common ligand is isolated in the absence of an expansion linker, the expansion linker can be subsequently attached to the common ligand, if desired.

The optimized common ligand module serves as a base structure onto which diversity can be built so that a variety of chemical moieties are oriented to allow binding to a specificity site of a receptor in the receptor family used to screen for an optimized binding ligand. The methods of the invention can further include the steps of screening the population of bi-ligands comprising the optimized common ligand module for binding to a first receptor in the receptor family; and identifying a bi-ligand having binding activity for the first receptor. The first receptor can be the same receptor used to screen for an optimized common ligand or can be another receptor in the same receptor family. Thus, the methods can be used to generate a population of bi-ligands suitable for screening a particular member of a receptor family as well as other members of the receptor family.

The following examples are intended to illustrate but not limit the present invention.

### EXAMPLE I

### Synthesis of a Pyridine Dicarboxylate Derivative Specificity Ligand and Expansion Linker

This example describes the synthesis of a pyridine dicarboxylate derivative that can function as a specificity ligand and expansion linker.

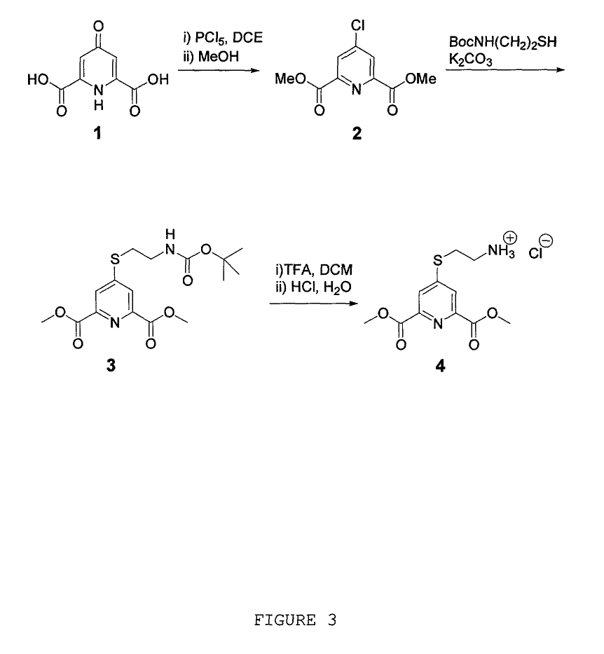

A specificity ligand and expansion linker attachment were synthesized as follows. FIG. 3 shows the reaction scheme for synthesis of a pyridine dicarboxylate derivative. For the synthesis of 4-chloro-pyridine-2,6-dicarboxylic acid dimethyl ester (compound 2), chelidamic acid monohydrate (compound 1) (10.0 g, 49.7 mmol) and phosphorous pentachloride were suspended in 250 ml of dichloroethane. The mixture was heated to 65° C. for 15 hours under an atmosphere of N₂. The colorless clear solution was cooled to 0° C. before 150 ml of methanol was added. The reaction mixture was stirred for another hour at 0° C. and was then neutralized with 4N NaOH solution at 0° C. The neutralized mixture was further buffered with saturated NaHCO₃ before the volatile solvents were removed in vacuo. The aqueous residue was diluted with H₂O and was extracted 4 times with EtOAc. The combined organic layers were dried over MgSO₄, filtered and concentrated in vacuo. The crude product was recrystallized from EtOAc and trace CH₂Cl₂ to afford 4-Chloro-pyridine-2,6-dicarboxylic acid dimethyl ester (compound 2) as a white solid (7.86 g, 68.9% yield).

NMR spectra were acquired on a Bruker Avance 300 spectrometer (Rheinstetten, Germany) at 300 MHz for ¹H and 75 MHz for ¹³C. Chemical shifts are recorded in parts per million (δ) relative to TMS (δ=0.0 ppm) for ¹H or to the residual signal of deuterated solvents (chloroform, δ=7.25 ppm for ¹H; δ=77.0 ppm for ¹³C) and coupling constant J is reported in Hz. The mass spectra were recorded on a Finnigan LCQ Duo apparatus (San Jose, Calif.) using APCI positive mode. For 4-chloro-pyridine-2,6-dicarboxylic acid dimethyl ester (compound 2), the NMR and mass spectra results were ¹H NMR (300 MHz, CD₃CD): δ 3.58 (s, 2H), 4.00 (s, 6H); MS m/z 230 (M+1).

For the synthesis of 4-(2-tert-butoxycarbonylamino-ethylsulfanyl)-pyridine-2,6-dicarboxylic acid dimethyl ester (compound 3; see FIG. 3), compound 2 (4-chloro-pyridine-2,6-dicarboxylic acid dimethyl ester) (1.54 g, 6.69 mmol) and K₂CO₃ (3 g, 24.19 mmol) were mixed in 50 ml of acetone and the resulting suspension was degassed by bubbling nitrogen for about 10 min. After adding neat N-Boc-thioethanolamine (1.24 ml, 7.34 mmol) through a syringe, the mixture was stirred at 50° C. under a nitrogen atmosphere for 5 hours and monitored by liquid chromatography/mass spetrometry (LC/MS). The mixture was filtered to remove solids and acetone was evaporated in vacuo. Dichloromethane was added and the organic phase washed with saturated aqueous NaHCO₃ (2×15 ml) and dried over MgSO₄. The solvent was removed in vacuo to afford compound 3 as a white solid (2.53 g, 100% yield). NMR spectra were acquired as described above. The NMR and mass spectra results for 4-(2-tert-butoxycarbonylamino-ethylsulfanyl)-pyridine-2,6-dicarboxylic acid dimethyl ester (compound 3) were: ¹H NMR (300 MHz, CDCl₃): δ 1.45 (s, 9H), 3.26 (t, J=6.5, 2H), 3.46 (q, J=6.4, 2H), 4.90 (br.s., 1H), 8.13 (s, 2H); MS m/z 371 (M+1).

For the synthesis of 4-(2-amino-ethylsulfanyl)-pyridine-2,6-dicarboxylic acid dimethyl ester (compound 4; FIG. 3), the free base was prepared as follows. 4-(2-tert-Butoxycarbonylamino-ethylsulfanyl)-pyridine-2,6-dicarboxylic acid dimethyl ester (compound 3) (6.69 mmol) was dissolved in 45 ml of dichloromethane and the resulting solution cooled under stirring to 0° C. Trifluoroacetic acid (TFA) (15 ml) was added drop wise and the mixture allowed to warm to room temperature over 5 hours, with LCMS monitoring. The solvent was removed in vacuo and TFA eliminated by three co-distillations with toluene (3×20 ml).

The resulting pasty solid was mixed with saturated aqueous NaHCO₃ and dichloromethane and stirred until gas evolution stopped. The two phases were separated and the aqueous phase extracted by dichloromethane (3×15 ml). After drying over MgSO₄, volatiles were removed by rotary evaporation providing compound 4 as a white powder (733 mg, 41% yield). The NMR and mass spectra results for 4-(2-amino-ethylsulfanyl)-pyridine-2,6-dicarboxylic acid dimethyl ester (compound 4) were: ¹H NMR (300 MHz, CDCl₃): δ 3.09 (t, J=6.1, 2H), 3.21 (t, J=6.2, 2H), 4.02 (s, 6H), 8.11 (s, 2H); MS m/z 271 (M+1).

For the preparation of the HCl salt, 4-(2-tert-butoxycarbonylamino-ethylsulfanyl)-pyridine-2,6-dicarboxylic acid dimethyl ester (compound 3) (6.54 mmol) was dissolved in 45 ml of dichloromethane and the solution cooled to 0° C. After adding neat trifluoroacetic acid (15 ml), the solution was stirred for 7 hours, with LCMS monitoring, while the temperature was allowed to reach 20° C. Volatiles were removed in vacuo and trifluoroacetic acid eliminated by co-distillation with toluene. The resulting pasty solid was then dissolved in 20 ml of 2N aqueous hydrochloric acid. Water was removed in vacuo, providing a white powder (2.33 g, 100% yield). The compound is hygroscopic and is stored in a well-closed flask. The NMR and mass spectra results for the HCl salt were: ¹H NMR (300 MHz, DMSO-d₆): δ 3.08 (m, 2H), 3.50 (m, 2H), 3.92 (s, 6H), 8.11 (s, 2H), 8.28 (br.s., 3H); ¹³C NMR (75.5 MHz, DMSO-d₆): δ 27.06, 37.35, 52.81, 124.14, 147.74, 150.73, 164.43; MS: m/z 271 (M).

These results describe the synthesis of a pyridine dicarboxylate derivative.

### EXAMPLE II

### Synthesis of Rhodanine and Thiazolidinedione Common Ligand Mimics and Biligands

This example describes the synthesis of rhodanine and thiazolidinedione derivatives.

FIG. 4 shows the reaction scheme for synthesis of rhodanine and thiazolidinedione bi-ligands. For the synthesis of 4-(5-formyl-furan-2-yl)-benzoic acid (compound 9a), 4-aminobenzoic acid (compound 5a) (60.0 g, 0.438 mol) was suspended in 100 ml of water. Under stirring, HCl 12M (225 ml) was added (exothermic) and the resulting suspension was stirred for about 10 min. The mixture was cooled to 1° C. A solution of NaNO₂ (30.2 g, 0.438 mol) in 200 ml of water was added in small portions, with addition time of 30 min, while maintaining the temperature between 5° C. and 10° C. The reaction mixture was stirred at 5° C. for an extra 30 min while adding an extra 300 ml of water, the reaction mixture still being a suspension.

A solution of CuCl₂.2H₂O (7.5 g, 0.044 mol) in 300 ml of water was added, followed by a pre-cooled solution of 2-furaldehyde (compound 6) (36 ml, 0.435 mol) in 50 ml of acetone. Under good stirring, 1.8 g of CuCl (0.018 mol) was added in small portions over a period of 10 min, resulting in foaming and precipitation of the expected compound. The ice bath was removed and the mixture stirred for 30 min, during which time the internal temperature increased from 5° C. to 15° C. An additional amount of 500 mg of CuCl (5 mmol) was added and the mixture stirred for 20 min, during which time the temperature increased to 20° C. An additional amount of 500 mg (5 mmol) of CuCl was added and the mixture stirred at room temperature for 16 hours.

The resulting brown precipitate was filtered, and thoroughly washed with water. After drying by lyophilization, the compound 9a was obtained as a brown powder (73.2 g, 77% mass yield). The purity of the material was about 70-80% according to NMR and it could be used for subsequent reaction without any further purification. A portion of compound 9a was purified by recrystallization in ethanol. The NMR results for 4-(5-formyl-furan-2-yl)-benzoic acid (compound 9a) were: ¹H NMR (300 MHz, DMSO-d₆): δ 7.31 (d, J=3.5, 1H), 7.66 (d, J=3.5, 1H), 7.82 (d, J=8.0, 2H), 8.00 (d, J=8.0, 2H), 9.62 (s, 1H).

For the synthesis of 3-(5-formyl-furan-2-yl)-benzoic acid (compound 9b; FIG. 4), 3-aminobenzoic acid (compound 5b) (60.0 g, 0.438 mol) was suspended in 400 ml of water. Under stirring, HCl 12 M (225 ml) was slowly added (exothermic) to form a thick suspension. NaNO₂ (30.2 g, 0.438 mol) as a solution in 200 ml of water was slowly added over a period of 20 min while maintaining the temperature between 10° C. and 15° C. with an ice bath and by shaking the suspension manually. The solid material progressively dissolved. The reaction mixture was stirred for an extra 30 min to reach 2° C. A solution of CuCl₂.2H₂O (7.5 g, 0.044 mol) in 300 ml of water was added, followed by a pre-cooled solution of 2-furaldehyde (compound 6) (36 ml, 0.435 mol) in 50 ml of acetone. The ice bath was removed and 1.8 g of CuCl was added under good stirring, in small portions resulting in foaming and precipitation of the expected compound. After 10 min. (T=13° C.), an extra amount of 500 mg of CuCl was added and the reaction stirred. Four extra 500 mg portions of CuCl were then added every 5 min (total amount of CuCl: 4.3 g, 0.043 mol), with the reaction re-staring upon each addition. The temperature after 35 min of reaction was 24° C. The mixture was stirred at room temperature for 17 hours and the precipitate filtered, washed twice with water and dried by lyophilization to afford a greenish brown solid (compound 9b) (65.5 g, 69% mass yield). According to NMR, the purity of the material was about 70-80% and could be used for subsequent reaction without any further purification. Some of compound 9b was purified by recristallization in ethanol. The NMR and mass spectra results for 3-(5-formyl-furan-2-yl)-benzoic acid (compound 9b) were: ¹H NMR (300 MHz, DMSO-d₆): δ 7.42 (d, J=3.43, 1H), 7.63-7.69 (m, 2H), 8.01 (d, J=7.6, 1H), 8.13 (d, J=7.7, 1H), 8.40 (s, 1H), 9.66 (s, 1H); MS: m/z 217 (M+1).

For the preparation of 5-(5-formyl-furan-2-yl)-2-hydroxy-benzoic acid methyl ester (compound 9c; FIG. 4), butyl lithium (BuLi) (105 mmol, 2.5 M in hexanes) was added to a solution of 4-methylpiperidine (10.00 g, 100 mmol) in 50 mL of tetrahydrofuran (THF) under N₂ at −78° C., followed by the addition of 2-furaldehyde (compound 6) (8.73 g, 91 mmol). The solution was kept at −78° C. for 15 min, and another portion of BuLi (105 mmol, 2.5 M solution in hexane) was added. The reaction mixture was allowed to warm to −20° C. and was stirred for 5 h. After being cooled to −78° C. Again, a solution of Me₃SnCl (100 mmol, 1 M solution in THF) was added to the reaction mixture. It was then allowed to warm gradually to room temperature and was stirred overnight. The reaction was quenched by adding 150 mL of cold brine and extracted with EtOAc (3×100 mL). The combined organic phase was dried and concentrated. Chromatography, in ethyl acetate (EtOAc)/Hexane 20:1, afforded 20.7 g (88.5%) of 5-trimethylstannanyl-furan-2-carbaldehyde (compound 8). The NMR and mass spectra results were: ¹H NMR (300 MHz, CDCl₃) δ 0.41 (s, 9H), 6.74 (d, J=3.7, 1H), 7.25 (d, J=3.6, 1H), 9.67 (s, 1H); MS m/z 261 (M+1).

As an alternative route of synthesis for compound 9° C., methy 2-hydroxy-5-bromobenzoate (compound 7) (2.30 g, 10 mmol), 5-trimethylstannanyl-furan-2-carbaldehyde (compound 8) (2.60 g, 10 mmol), and tetrakis (triphenylphosphine)palladium (0.577 g, 1 mmol) in 25 mL of dimethylformamide (DMF)was heated at 60° C. under N₂ atmosphere for 30 h. The solution was evaporated to dryness under reduce pressure and the residue was purified by chromatography (EtOAc/hexane 1:1) to give 2.13 g (86.2%) of methyl 5-(5-formyl-furan-2-yl)-2-hydroxy-benzoic acid methyl ester (compound 9c). The NMR and mass spectra results for 5-(5-formyl-furan-2-yl)-2-hydroxy-benzoic acid methyl ester (compound 9c) were: ¹H NMR (300 MHz, CDCl₃) δ 4.03 (s, 3H), 6.78 (d, J=3.2, 1H), 7.10 (d, J=8.8, 1H), 7.27 (s, 1H), 7.34 (d, J=2.2, 1H), 7.92 (d, J=8.6, 1H), 8.36 (s, 1H), 9.64 (s, 1H), 11.03 (s, 1H); MS m/z 247 (M+1).

For the synthesis of 4-[5-(2,4-dioxo-thiazolidin-5-ylidenemethyl)-furan-2-yl]-benzoic acid (compound 11a; FIG. 4), crude aldehyde (compound 9a) (30.2 g, about 0.140 mol) and 2,4-thiazolidinedione (compound 10a) (18.0 g, 0.154 mol) were mixed in 500 ml of ethanol in a 1 L flask equipped with a magnetic stirring bar. Piperidine (2.8 ml, 0.028 mol) was added, and the resulting suspension was heated at 70° C. under good stirring for 5 hours. The mixture was cooled down with ice and the yellow precipitate was collected and washed with ethyl acetate and ether. In order to eliminate remaining piperidine (about 10%), the crude product was suspended in 100 ml of aqueous HCl 0.1N and placed in an ultrasound bath for 10 min. After filtration and drying by lyophilization, compound 11a was obtained as a yellow orange powder (16.95 g, 38% yield). The NMR and mass spectra results for 4-[5-(2,4-dioxo-thiazolidin-5-ylidenemethyl)-furan-2-yl]-benzoic acid (compound 11a) were: ¹H NMR (300 MHz, DMSO-d₆): δ 7.24 (d, J=3.6, 1H), 7.40 (d, J=3.6, 1H), 7.63 (s, 1H ), 7.89 (d, J=8.2, 2H), 8.06 (d, J=8.3, 2H); ¹³C NMR (75.5 MHz, DMSO-d₆): δ 111.46, 117.67, 120.87, 121.06, 124.03, 130.18, 130.40, 132.36, 149.68, 155.58, 166.75, 166.92, 168.57; MS m/z 316 (M+1).

For the synthesis of 4-[5-(4-oxo-2-thioxo-thiazolidin-5-ylidenemethyl)-furan-2-yl]-benzoic acid (compound 11b; FIG. 4), 4-(5-Formyl-furan-2-yl)-benzoic acid (compound 9a) (412 mg, 1.91 mmol), rhodanine (compound 10b) (279 mg, 2.09 mmol) and piperidine (38 μl, 0.384 mmol) were placed in 5 ml of ethanol in a vial. The mixture was stirred under microwave irradiation for 300 s at 160° C. After cooling down to room temperature, the orange precipitate was filtered, washed with ethyl acetate and ether and dried in vacuo to provide (compound 11b) as an orange powder (477 mg, 75% yield). The NMR and mass spectra results for 4-[5-(4-Oxo-2-thioxo-thiazolidin-5-ylidenemethyl)-furan-2-yl]-benzoic acid (compound 11b) were: ¹H NMR (300 MHz, DMSO-d₆): δ 7.34 (d, J=3.3, 1H), 7.45 (d, J=3.2, 1H), 7.52 (s, 1H), 7.93 (d, J=8.2, 2H) and 8.08 (d, J=8.0, 2H); MS: m/z 332 (M+1).

For the synthesis of the 3-[5-(2,4-dioxo-thiazolidin-5-ylidenemethyl)-furan-2-yl]-benzoic acid common ligand (compound 11c; FIG. 4), the crude aldehyde (compound 9b) (35.0 g, 0.162 mol) and 2,4-thiazolidinedione (compound 10a) (22.8 g, 0.195 mol) were mixed in 500 ml of ethanol in a 1L flask equipped with a magnetic stirring bar. Piperidine (1.6 ml, 0.0162 mol) was added through syringe and the suspension was heated at 70° C. under good stirring for 5 hours. The mixture was cooled down with ice and the yellow precipitate was collected and washed with ethyl acetate and ether. In order to eliminate remaining piperidine (about 10%), the crude product was suspended in 100 ml of aqueous HCl 0.1 N and placed in an ultrasound bath for 10 min. After filtration and drying by lyophilization, compound 11c was obtained as a nice yellow-orange powder (18.51 g, 36% yield). The NMR and mass spectra results for 3-[5-(2,4-dioxo-thiazolidin-5-ylidenemethyl)-furan-2-yl]-benzoic acid (compound 11c) were: ¹H NMR (300 MHz, DMSO-d₆): δ 7.22 (d, J=3.4, 1H), 7.39 (d, J=3.4, 1H), 7.63 (s, 1H), 7.66 (t, J=7.8, 1H), 7.96 (d, J=7.3, 1H), 8.05 (d, J=7.7, 1H), 8.37 (s, 1H); ¹³C NMR (75.5 MHz, DMSO-d₆): δ 110.31, 117.72, 120.81, 120.86, 124.64, 128.22, 129.16, 129.39, 129.64, 131.82, 149.24, 155.68, 166.78, 167.26, 168.76; MS m/z 316 (M).

For the synthesis of the 3-[5-(4-oxo-2-thioxo-thiazolidin-5-ylidenemethyl)-furan-2-yl]-benzoic acid common ligand (compound 11d; FIG. 4), the compound 9b (3.45 mmol), rhodanine (compound 10b) (460 mg, 3.45 mmol), water (15 ml) and ethanolamine (21 μL, 0.35 mmol) were placed in a flask. The suspension was stirred at 90° C. for 3 hours. After cooling down to room temperature, the orange precipitate was filtered and dried in vacuo to give compound 11d (573 mg, 50%). The NMR and mass spectra results for 3-[5-(4-oxo-2-thioxo-thiazolidin-5-ylidenemethyl)-furan-2-yl]-benzoic acid (compound 11d) were: ¹H NMR (300 MHz, DMSO-d₆): δ 7.31 (d, J=3.6, 1H), 7.43 (d, J=3.6, 1H), 7.50 (s, 1H ), 7.69 (t, J=7.8, 1H), 7.97 (d, J=7.7, 1H), 8.07 (d, J=7.8, 1H), 8.38 (s, 1H).

For the synthesis of the 5-[5-(2,4-dioxo-thiazolidin-5-ylidenemethyl)-furan-2-yl]-2-hydroxy-benzoic acid methyl ester common ligand (compound 11e; FIG. 4), 2,4-Thiazolidinedione (compound 10a) (539 mg, 4.60 mmol) and compound 9c (872 mg, 3.54 mmol) were suspended in 25 mL of ethanol. Five drops of piperidine were added and the mixture was heated at 70° C. for 5 hours. The reaction was then cooled to room temperature overnight and the bright orange precipitate was collected on a fritted filter to give 1.1 g (90%) of compound 11e. The NMR and mass spectra results for 5-[5-(2,4-Dioxo-thiazolidin-5-ylidenemethyl)-furan-2-yl]-2-hydroxy-benzoic acid methyl ester (compound 11e) were: 1H NMR (300 MHz, DMSO-d₆): δ 3.93 (s, 3H), 7.14 (d, J=8.7, 1H), 7.19 (m, 2H), 7.61 (s, 1H ), 7.92 (d, J=2.3, 8.7, 1H), 8.16 (d, J=2.3, 1H), 10.71 (s, 1H).

For the synthesis of the 5-[5-(2,4-dioxo-thiazolidin-5-ylidenemethyl)-furan-2-yl]-2-hydroxy-benzoic acid common ligand (compound 11f; FIG. 4), compound 11e (500 mg, 1.45 mmol) was suspended in methanol. A solution of lithium hydroxide (LiOH) (800 mg, 16.7 mmol) in 8 mL of H₂O was added. The reaction mixture was stirred at room temperature for 20 hours. The clear solution was then acidified with 2N HCl to pH 1 and was quickly extracted three times with EtOAc. The combined organic layers were dried over MgSO₄, filtered and concentrated in vacuo to give 450 mg (94% yield) of compound 11f. The NMR and mass spectra results for 5-[5-(2,4-dioxo-thiazolidin-5-ylidenemethyl)-furan-2-yl]-2-hydroxy-benzoic acid (compound 11f) were: ¹H NMR (300 MHz, DMSO-d₆): δ 6.76 (d, J=8.5, 1H), 6.96 (d, J=3.7, 1H), 7.14 (d, J=3.7, 1H), 7.54 (s, 1H), 7.63 (dd, J=8.5, 2.4, 1H), 8.14 (d, J=2.4, 1H).

For the synthesis of the 4-(2-{4-[5-(2,4-dioxo-thiazolidin-5-ylidenemethyl)-furan-2-yl]-benzoylamino}-ethylsufanyl)-pyridine-2,6-dicarboxylic acid (compound 12a), compound 4 (free base, 75 mg, 0.277 mmol), compound 11a (87 mg, 0.276 mg) and hydroxybenzotriazole hydrate (HOBt.H₂O) (51 mg, 0.333 mmol) were dissolved in DMF (1 ml). After adding triethylamine (46 ml, 0.331 mmol) and 1-dimethylaminopropyl-3-ethyl-carbodiimide (EDCI) (70 mg, 0.333 mmol), the mixture was stirred at room temperature for 24 hours. The resulting precipitate (52.4 mg) was collected on a funnel, washed with DMF, aqueous 0.5 N HCl and methanol (MeOH). 48.2 mg of the solid was suspended in MeOH (0.5 ml) and water (0.5 ml) before adding LiOH (14 mg, 0.585 mmol). After stirring at room temperature for 1.5 hours, the homogenous solution was acidified with aqueous 2N HCl and the precipitate filtered, washed with water and dried. The reaction afforded a bright yellow solid (compound 12a) (41.5 mg, 30% yield). The NMR and mass spectra results for 4-(2-{4-[5-(2,4-dioxo-thiazolidin-5-ylidenemethyl)-furan-2-yl]-benzoylamino}-ethylsufanyl)-pyridine-2,6-dicarboxylic acid (compound 12a) were: ¹H NMR (300 MHz, DMSO-d₆): δ 3.42 (m, 2H), 3.60 (m, 2H), 7.26 (d, J=3.6, 1H), 7.41 (d, J=3.5, 1H), 7.67 (s, 1H), 7.89 (d, J=8.3, 2H), 7.95 (d, J=8.4, 2H), 8.08 (s, 2H, ), 8.85 (br. t., 1H); MS m/z 540 (M+1).

For the synthesis of the 4-(2-{4-[5-(4-oxo-2-thioxo-thiazolidin-5-ylidenemethyl)-furan-2-yl]-benzoylamino}-ethylsulfanyl)-pyridine-2,6-dicarboxylic acid biligand (compound 12b; FIG. 4), compound 4 (HCl salt, 84 mg, 0.275 mmol), compound 11b (91 mg, 0.275 mmol) and HOBt.H₂O (51 mg, 0.333 mmol) were dissolved in DMF (1 ml). After adding triethylamine (0.11 ml, 0.79 mmol) and EDCI (0.329 mmol), the mixture was stirred at room temperature for 24 hours. Addition of four drops of concentrated HCl induced formation of a precipitate (159 mg) which was filtered and washed with aqueous 0.1 N HCl and dried in vacuo. 111 mg of this compound was placed in water (0.5 ml) and MeOH (0.5 ml). After addition of LiOH (40 mg, 1.67 mmol), the solution was stirred at room temperature for 2 hours. The lithium salt of the expected compound, which precipitated, was then isolated by filtration. The salt could be dissolved in warm water (about 40° C.) and precipitated by addition of aqueous 2N Hcl. After filtration and drying in vacuo, compound 12b was obtained as a red powder (41 mg, 38% yield). The NMR and mass spectra results for 4-(2-{4-[5-(4-oxo-2-thioxo-thiazolidin-5-ylidenemethyl)-furan-2-yl]-benzoylamino}-ethylsulfanyl)-pyridine-2,6-dicarboxylic acid (compound 12b) were: ¹H NMR (300 MHz, DMSO-d₆): δ 3.54 (br. t., 2H), 3.60 (br. t., 2H), 7.35 (d, J=3.5, 1H), 7.44 (d, J=3.5, 1H), 7.54 (s, 1H), 7.91 (d, J=8.2, 2H), 7.99 (d, J=8.3, 2H), 8.08 (s, 2H), 8.87 (br. t., 1H); MS m/z 556 (M+1).

For the synthesis of the 4-(2-{3-[5-(2,4-dioxo-thiazolidin-5-ylidenemethyl)-furan-2-yl]-benzoylamino}-ethylsulfanyl)-pyridine-2,6-dicarboxylic acid biligand (compound 12c), compound 4 (HCl salt, 100 mg, 0.326 mmol), compound 11c (103 mg, 0.327 mmol) and HOBt.H₂O (60 mg, 0.392 mmol) were dissolved in DMF (1 ml). After adding triethylamine (0.14 ml, 1.01 mmol) and EDCI (75 mg, 0.391 mmol), the solution was stirred at room temperature for 2.5 days. The resulting solid (73 mg) was collected on a funnel, washed with aqueous 0.5 N HCl and dried. 63 mg of the product was suspended in water (0.5 ml) and MeOH (0.5 ml) before adding LiOH (20 mg, 0.84 mmol). After stirring at room temperature for 1.5 hours, water was added and the compound precipitated by acidification with aqueous 2N HCl. After drying in vacuo, a pure compound 12c was obtained as a yellow powder (49 mg, 32% yield). The NMR and mass spectra results for 4-(2-{3-[5-(2,4-Dioxo-thiazolidin-5-ylidenemethyl)-furan-2-yl]-benzoylamino}-ethylsulfanyl)-pyridine-2,6-dicarboxylic acid (compound 12c) were: ¹H NMR (300 MHz, DMSO-d₆): δ 3.62 (br. m., 2H) and one signal overlapped by water at 3.44, 7.25 (d, J=3.5, 1H), 7.33 (d, J=3.5, 1H), 7.62 (t, J=7.8, 1H), 7.67 (s, 1H), 7.81 (d, J=7.7, 1H), 7.95 (d, J=7.7, 1H), 8.08 (s, 2H), 8.24 (s, 1H), 8.91 (br. t., 1H); MS m/z 540 (M+1).

For the synthesis of the 4-(2-{3-[5-(4-oxo-2-thioxo-thiazolidin-5-ylidenemethyl)-furan-2-yl]-benzoylamino}-ethylsulfanyl)-pyridine-2,6-dicarboxylic acid biligand (compound 12d; FIG. 4), compound 4 (free base, 80 mg, 0.296 mmol), compound 11d (98 mg, 0.296 mmol) and HOBt.H₂O (54 mg, 0.353 mmol) were dissolved in DMF (1 ml). After adding triethylamine (49 μl, 0.352 mmol) and EDCI (72 mg, 0.375 mmol), the solution was stirred at room temperature for 30 hours. The resulting orange precipitate (95 mg) was filtered, washed with DMF and aqueous 0.5 N HCl and dried. 88.2 mg of the compound was suspended in water (1 ml) and MeOH (1 ml) before adding LiOH (25 mg, 1.05 mmol). After stirring at room temperature for 2.5 hours, the solution was acidified with aqueous 2N HCl and the resulting solid filtered and washed with water. After drying, compound 12d was as a red powder (65 mg, 42%). The NMR and mass spectra results for 4-(2-{3-[5-(4-oxo-2-thioxo-thiazolidin-5-ylidenemethyl)-furan-2-yl]-benzoylamino}-ethylsulfanyl)-pyridine-2,6-dicarboxylic acid (compound 12d) were: ¹H NMR (300 MHz, DMSO-d₆): δ 3.63 (m, 2H) and one signal overlapped by water at 3.39, 7.35 (s, 2H), 7.55 (s, 1H ), 7.63 (t, J=7.7, 1H), 7.82 (d, J=7.7, 1H), 7.97 (d, J=7.7, 1H), 8.08 (s, 2H), 8.27 (s, 1H), 8.93 (br. t., J=5.1, 1H); MS m/z 556 (M+1).

For the synthesis of the 4-(2-{5-[5-(2,4-dioxo-thiazolidin-5-ylidenemethyl)-furan-2-yl]-2-hydroxy-benzoylamino}-ethylsulfanyl)-pyridine-2,6-dicarboxylic acid biligand (compound 12f; FIG. 4), compound 4 (free base, 73 mg, 0.270 mmol), compound 11f (89 mg, 0.269 mmol) and HOBt.H₂O (49 mg, 0.320 mmol) were dissolved in DMF (1 ml). After adding triethylamine (45 ml, 0.324 mmol) and EDCI (62 mg, 0.323 mmol), the mixture was stirred at room temperature for 30 hours. The reaction was acidified with HCl inducing formation of an orange precipitate that was isolated by filtration. The compound was purified by flash chromatography (SiO₂, MeOH 5% to 7.5% in dichloromethane) and suspended in a mixture of MeOH (0.5 ml) and water (0.5 ml). After adding LiOH (15 mg) and stirring for 2 hours at room temperature, the homogenous solution was acidified by aqueous 2N Hcl. The resulting compound was filtered and purified by preparative HPLC to give a reddish powder for compound 12f (16.1 mg, 15% yield). The NMR and mass spectra results for 4-(2-{5-[5-(2,4-dioxo-thiazolidin-5-ylidenemethyl)-furan-2-yl]-2-hydroxy-benzoylamino}-ethylsulfanyl)-pyridine-2,6-dicarboxylic acid (compound 12f) were: ¹H NMR (300 MHz, DMSO-d₆): δ 3.66 (m, 2H) and signal overlapped by water at 3.37, 7.10 (m, 2H), 7.22 (d, J=3.0, 1H), 7.63 (s, 1H), 7.81 (d, J=8.1, 1H), 8.11 (s, 2H), 8.24 (s, 1H), 9.12 (br. t., 1H); MS m/z 468 (M+H−2CO₂).

This example describes the synthesis of rhodanine and thiazolidinedione derivatives as common ligand mimics.

### EXAMPLE III

### Synthesis of Pseudothiohydantoin Common Ligand Mimics and Biligands

This example describes the synthesis of pseudothiohydantoin derivatives.

FIG. 5 shows the reaction scheme for the synthesis of pseudothiohydantoin derivatives. For the synthesis of 4-(2-imino-4-oxo-thiazolidin-5-ylidenemethyl)-benzoic acid (compound 15c), pseudothiohydantoin (compound 14) (116 mg, 1 mmol) and 4-carboxybenzaldehyde (compound 13c) (1 mmol) were suspended in acetic acid (3 ml). The mixture was heated at 95° C. for 8 hours. It was then cooled to room temperature. The solid was then collected and washed with water, ethyl acetate to give 15c as a solid (215 mg, 0.89 mmol, 89 %). NMR spectra were acquired as described above. The resulting data for 4-(2-Imino-4-oxo-thiazolidin-5-ylidenemethyl)-benzoic acid (compound 15c) was : ¹H NMR (300 MHz, DMSO-d₆): d 764-7.70 (m, 2H), 7.70 (s, 1H), 8.03-8.05 (m, 2H).

2-Hydroxy-5-(2-imino-4-oxo-thiazolidin-5-ylidenemethyl)-benzoic acid (compound 15a) and 3-(2-Imino-4-oxo-thiazolidin-5-ylidenemethyl)-benzoic acid (compound 15b) were prepared from compounds 13a and 13b, respectively, as shown in FIG. 5 and described for compound 15c. Compound 15a was obtained at 72% yield. The NMR and mass spectra results for 2-hydroxy-5-(2-imino-4-oxo-thiazolidin-5-ylidenemethyl)-benzoic acid (compound 15a) were: ¹H NMR (300 MHz, DMSO-d₆): δ 7.08 (d, J=8.4, 1H), 7.56 (s, 1H), 7.76 (d, J=8.4, 1H), 8.04 (s, 1H), 9.11 (s, 1H); MS: m/z 265 (M+1). Compound 15b was obtained at 81% yield. The NMR and mass spectra results for 3-(2-imino-4-oxo-thiazolidin-5-ylidenemethyl)-benzoic acid (compound 15b) were: ¹H NMR (300 MHz, DMSO-d₆): δ 7.61-7.66 (m, 1H), 7.66 (s, 1H), 7.84-7.86 (m, 1H), 7.95-7.98 (m, 1H), 8.17 (s, 1H).

For the synthesis of the 4-{2-[2-hydroxy-5-(2-imino-4-oxo-thiazolidin-5-ylidenemethyl)-benzoylamino]-ethylsulfanyl}-pyridine-2,6-dicarboxylic acid (compound 16a), compound 4 (free base, 77 mg, 0.284 mmol), compound 15a (75 mg, 0.284 mmol) and HOBt.H₂O (52 mg, 0.340 mmol) were dissolved in 1 ml of DMF. Triethylamine (47 ml, 0.338 mmol) and EDCI (72 mg, 0.375 mmol) were added and the resulting mixture stirred at room temperature for 17 hours. The precipitate (39 mg) was collected on a funnel and washed with a bit of DMF and aqueous 2N HCl. This crude intermediate (37 mg) was suspended in water (0.5 ml) and MeOH (0.5 ml). After adding LiOH (12 mg, 0.50 mmol) and stirring at room temperature for 2 hours, the reaction mixture turned homogenous. Precipitation of the compound was achieved by adding 2N aqueous HCl. After filtration and drying, compound 16a was isolated as a yellow solid (26.2 mg, 20% yield). The NMR and mass spectra results for 4-{2-[2-hydroxy-5-(2-imino-4-oxo-thiazolidin-5-ylidenemethyl)-benzoylamino]-ethylsulfanyl}-pyridine-2,6-dicarboxylic acid (compound 16a) were: ¹H NMR (300 MHz, DMSO-d₆): δ 3.44 (m, 2H), 3.65 (m, 2H), 7.05 (d, J=8.6, 1H), 7.57 (d, J=7.1, 1H), 7.49 (s, 1H), 8.07 (s, 3H), 9.12 (br. s., 1H), 9.40 (br. s., 1H); MS m/z 489 (M+1).

For the synthesis of the 4-{2-[3-(2-imino-4-oxo-thiazolidin-5-ylidenemethyl)-benzoylamino]-ethylsulfanyl}-pyridine-2,6-dicarboxylic acid biligand (compound 16b; FIG. 5), compound 4 (free base, 88 mg, 0.326 mmol), pseudothiohydanthoin (compound 15b) (81 mg, 0.326 mmol) and HOBt.H₂O (60 mg, 0.392 mmol) were suspended in DMF (2 ml). After addition of triethylamine (54 ml, 0.388 mmol) and EDCI (75 mg, 0.391 mmol), the suspension was well stirred for 2.5 days. The resulting precipitate (41 mg) was collected on a funnel and washed with a bit of DMF and aqueous 0.5 N HCl. The crude compound (37.3 mg) was then suspended in MeOH (0.5 ml) and water (0.5 ml) before LiOH (16 mg, 0.668 mmol) was added. After stirring for 1.5 hours (homogenous), the mixture was acidified with aqueous 2N HCl. The precipitate was collected, washed with water and dried to afford compound 16b as a pale yellow powder (32.5 mg, 92% yield). The NMR and mass spectra results for 4-{2-[3-(2-imino-4-oxo-thiazolidin-5-ylidenemethyl)-benzoylamino]-ethylsulfanyl}-pyridine-2,6-dicarboxylic acid (compound 16b) were: ¹H NMR (300 MHz, DMSO-d₆): δ 3.43 (m, 2H), 3.60 (m, 2H), 7.59 (t, J=7.7, 1H), 7.62 (s, 1H), 7.73 (d, J=7.7, 1H), 7.84 (d, J=7.6, 1H), 8.05 (s, 1H), 8.07 (s, 2H), 8.91 (br. t., J=5.0, 1H), 9.32 (br. s., 1H); MS m/z 385 (M+H−CO₂).

For the synthesis of the 4-{2-[4-(2-imino-4-oxo-thiazolidin-5-ylidenemethyl)-benzoylamino]-ethylsulfanyl}-pyridine-2,6-dicarboxylic acid biligand (compound 16c; FIG. 5), compound 4 (HCl salt, 102 mg, 0.332 mmol), compound 15c (83 mg, 0.334 mmol) and HOBt.H₂O (61 mg, 0.398 mmol) were placed in 2 ml of DMF. After adding triethylamine (0.14 ml, 1.01 mmol) and EDCI (76 mg, 0.396 mmol), the mixture was stirred at room temperature for 2 days. The resulting pale yellow precipitate (94 mg) was filtered and washed with aqueous 2N HCl. 78 mg of that compound was suspended in water (0.5 ml) and MeOH (0.5 ml). LiOH (26 mg, 0.96 mmol) was added and the mixture stirred at room temperature for 2.5 hours. The mixture was acidified with aqueous 2N HCl and the product collected on a funnel. The remaining triethylamine (about 20%) was eliminated by ultrasound for 30 min in aqueous HCl and filtration to afford a yellow powder (compound 16c) (41 mg, 32% yield). The NMR and mass spectra results for 4-{2-[4-(2-imino-4-oxo-thiazolidin-5-ylidenemethyl)-benzoylamino]-ethylsulfanyl}-pyridine-2,6-dicarboxylic acid (compound 16c) were: ¹H NMR (300 MHz, DMSO-d₆): δ 3.58 (t, J=5.5, 2H) and one signal overlapped by water, 7.63 (s, 1H), 7.64 (d, J=9.7, 2H), 7.92 (d, J=8.1, 2H), 8.07 (s, 2H), 8.88 (br. t., J=5.1, 1H), 9.26 (br. s., 1H) and 9.53 (br. s., 1H); MS m/z 473 (M+1).

This example describes the synthesis of pseudothiohydantoin as common ligand mimics.

### EXAMPLE IV

### Synthesis of Benzimidazole Common Ligand Mimics and Bi-ligands

This example describes synthesis of benzimidazole derivatives.

FIG. 6 shows the reaction scheme for synthesis of benzimidazole derivatives. For the synthesis of 4-(5-formyl-furan-2-yl)-benzoic acid methyl ester (compound 18), a mixture of methy 4-bromobenzoate (compound 17) (2.15 g, 10 mmol), 5-trimethylstannanyl-furan-2-carbaldehyde (compound 8), and tetrakis (triphenylphosphine) palladium (0.577 g, 1 mmol) in 20 mL of DMF was heated under N₂ at 60° C. for 20 h. The solution was evaporated to dryness under reduced pressure and the residue was purified by chromatography (EtOAc/hexane 1:3) to give 2.185 g (95% yield) of 4-(5-formyl-furan-2-yl)-benzoic acid methyl ester (compound 18). The NMR and mass spectra results for 4-(5-formyl-furan-2-yl)-benzoic acid methyl ester (compound 18) were: ¹H NMR (300 MHz, CDCl₃) δ 3.98 (s, 3H), 6.97 (d, J=3.8, 1H ), 7.36 (d, J=3.8, 1H), 7.91 (d, J=6.8, 2H), 8.14 (d, J=6.8, 2H), 9.72 (s, 1 H); MS m/z 231 (M+1).

For the synthesis of the 4-[5-(5-nitro-1H-benzoimidazol-2-yl)-furan-2-yl]-benzoic acid common ligand mimic (compound 20a), a solution of methyl 4-(5-formyl-furan-2-yl)-benzoic acid methyl ester (compound 18) (115 mg, 0.50 mmol), 4-nitro-benzene-1,2-diamine (77 mg, 0.50 mmol) and benzoquinone (54 mg, 0.50 mmol) in 10 mL of ethanol was heated to reflux for 4 h. The solvent was removed and the residue was dissolved in 50 ml of CH₂Cl₂, washed with brine (2×10 mL). Concentration and flash chromatography purification of the residue in EtOAC/Hexane 1:1 gave 4-[5-(5-nitro-1H-benzoimidazol-2-yl)-furan-2-yl]-benzoic methyl ester as a crude product. The benzimidazole ester was dissolved in a mixture of ethanol (5 ml) and 10% KOH (5 mL). After being heated to reflux for 3 h, the reaction mixture was poured into 1N HCl (30 mL) and extracted with EtOAc (3×10 mL). The combined organic phase was dried and concentrated and HPLC purified to give a solid product (compound 20a) (118 mg, 0.34 mmol, 68% yield). The NMR and mass spectra results for 4-[5-(5-nitro-1H-benzoimidazol-2-yl)-furan-2-yl]-benzoic acid (20a) were: ¹H NMR (300 MHz, CD₃OD) δ 7.24 (d , J=3.8, 1H), 7.46 (d, J=3.7, 1H), 7.76 (d, J=8.91, 1H), 8.02 (d, J=8.5, 2H), 8.14 (d, J=8.5, 2H), 8.29 (dd, J=2.1, 5.8, 1 H), 8.52 (d, J=2.1, 1H); MS m/z 350 (M+1).

4-[5-(1H-Benzoimidazol-2-yl)-furan-2-yl]-benzoic acid (compound 20b) was prepared in 91% overall yield using the procedure to prepare compound 20a (see FIG. 6). The NMR and mass spectra results for 4-[5-(1H-benzoimidazol-2-yl)-furan-2-yl]-benzoic acid (compound 20b) were: ¹H NMR (300 MHz, CD₃OD) δ 7.28 (dd, J=3.0, 6.2, 2H), 7.4 (m, 2H), 7.65 (dd, J=3.1, 6.0, 2H), 8.06 (s, 1H); MS m/z 305 (M+1).

For the synthesis of the 4-(2-{4-[5-(5-nitro-1H-benzoimidazol-2-yl)-furan-2-yl]-benzoylamino}-ethylsulfanyl)-pyridine-2,6-dicarboxylic acid biligand (compound 21a; FIG. 6), compound 4 (free base, 32 mg, 0.118 mmol), compound 20a (41 mg, 0.117 mmol) and HOBt.H₂O (21 mg, 0.137 mmol) were dissolved in DMF (1 ml). Triethylamine (20 μl, 0.144 mmol) and EDCI (27 mg, 0.141 mmol) were added and the solution stirred at room temperature for 31 hours. Addition of aqueous 2N HCl induced formation of a precipitate (53 mg) which were isolated by filtration and washed with aqueous 0.5N HCl. 48 mg of that compound was mixed with water (0.5 ml), MeOH (0.5 ml) and LiOH (15 mg, 0.63 mmol) and the suspension stirred at room temperature for 4 hours. The compound was precipitated by adding aqueous 2N HCl. After filtration and drying, compound 21a was isolated as a brown powder (43 mg, 93% yield). The NMR and mass spectra results for 4-(2-{4-[5-(5-nitro-1H-benzoimidazol-2-yl)-furan-2-yl]-benzoylamino}-ethylsulfanyl)-pyridine-2,6-dicarboxylic acid (compound 21a) were: ¹H NMR (300 MHz, DMSO-d₆): δ 3.44 (m, 2H), 3.63 (m, 2H), 7.42 (d, J=3.4, 1H), 7.53 (d, J=3.3, 1H), 7.80 (d, J=8.9, 1H), 7.97 (d, J=8.2, 2H), 8.05 (d, J=8.1, 2H), 8.09 (s, 2H), 8.17 (dd, J=8.8, 1.7, 1H), 8.49 (s, 1H), 8.89 (br. s., 1H), 8.30 (br. s., 1H); MS m/z 574 (M+1).

For the synthesis of the 4-(2-{4-[5-(1H-Benzoimidazol-2-yl)-furan-2-yl]-benzoylamino}-ethylsulfanyl)-pyridine-2,6-dicarboxylic acid biligand (compound 21b; FIG. 6), compound 4 (HCl salt) (50 mg, 0.163 mmol), compound 20b (49.8 mg, 0.164 mmol) and HOBt.H₂O (30 mg, 0.196 mmol) were dissolved in DMF (1 ml). After adding triethylamine (68 μl, 0.489 mmol) and EDCI (38 mg, 0.198 mmol), the reaction mixture was stirred at room temperature for 16 hours. Upon acidification with aqueous 2N HCl, a brown precipitate (76 mg) was formed and isolated by filtration. The brown product (69 mg) was mixed with water (0.5 ml), MeOH (0.5 ml) and LiOH (21 mg, 0.88 mmol) and the resulting mixture stirred at room temperature for 1.5 hours. Addition of aqueous 2N HCl and filtration afforded 52 mg (66% yield) of crude product (about 90% pure). Purification by preparative HPLC provided 2.7 mg of pure compound 21b. The NMR and mass spectra results for 4-(2-{4-[5-(1H-benzoimidazol-2-yl)-furan-2-yl]-benzoylamino}-ethylsulfanyl)-pyridine-2,6-dicarboxylic acid (compound 21b) were: ¹H NMR (300 MHz, DMSO-d₆): δ 3.61 (m, 2H) and one signal overlapped by water, 7.27 (m, 2H), 7.63 (m, 2H), 7.36 (s, 2H), 7.95 (d, J=8.2, 2H), 8.01 (d, J=8.3, 2H), 8.09 (s, 2H), 8.86 (br. t., 1H); MS m/z 441 (M+H−2CO₂).

This example describes the synthesis of benzimidazole derivatives.

### EXAMPLE V

### Synthesis of Additional Pyridine Dicarboxylate Derivatives

This example describes the synthesis of additional pyridine dicarboxylate derivatives.

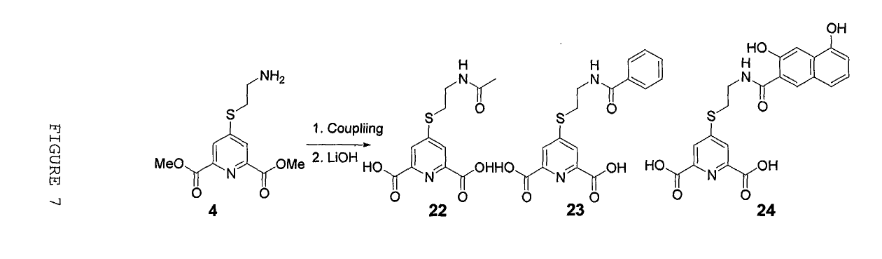

FIG. 7 shows the reaction scheme for pyridine carboxylate derivatives. For the synthesis of 4-(2-acetylamino-ethylsulfanyl)-pyridine-2,6-dicarboxylic acid (compound 22), compound 4 (HCl salt) (113.2 mg, 0.369 mmol) was dissolved in dichloromethane (1 ml) and the mixture cooled to 0° C. Upon addition of triethylamine (0.12 ml, 0.86 mmol), a white precipitate was formed. Acetyl chloride (32 ml, 0.45 mmol) and dichloromethane (1 ml) were added and the solution stirred at 0° C. for 5 min and at room temperature for 1 hour. After adding aqueous 0.1 N HCl (25 ml), the compound was extracted with dichloromethane (3×10 ml). The combined extracts were washed with saturated aqueous NaHCO₃ (15 ml), dried over MgSO₄ and the volatiles removed in vacuo to provide a white solid. The compound was then mixed with water (1 ml), MeOH (1 ml) and LiOH (36 mg, 1.5 mmol) and the mixture stirred at room temperature for 1.25 hours. Aqueous 2N HCl and water were added and the solution cooled in an ice bath resulting in formation of a precipitate. Filtration and drying in vacuo provided the expected compound 22 as a white powder (27 mg, 25% yield). The NMR and mass spectra results for 4-(2-acetylamino-ethylsulfanyl)-pyridine-2,6-dicarboxylic acid (compound 22) were: ¹H NMR (300 MHz, DMSO-d₆): δ 1.79 (s, 3H), 3.27 (m, 2H) and 3.32 (m, 2H), 8.03 (s, 2H), 8.15 (br. t., 1H); MS m/z 285 (M+1).

For the synthesis of 4-(2-benzoylamino-ethylsulfanyl)-pyridine-2,6-dicarboxylic acid (compound 23), compound 4 (HCl salt) (90 mg, 0.293 mmol), benzoic acid (36 mg , 0.295 mmol) and HOBt.H₂O (54 mg, 0.353 mmol) were dissolved in DMF (1 ml). After adding triethylamine (0.14 ml, 1.01 mmol) and EDCI (67 mg, 0.350 mmol), the mixture was stirred at room temperature for 21 hours. Aqueous 2N HCl and water were added and the solution was extracted with dichloromethane (4×15 ml). The combined organic extracts were washed with saturated aqueous NaHCO₃ (2×10 ml), dried over MgSO₄ and the solvent removed in vacuo to afford a white solid. The compound was placed in a mixture of MeOH (1 ml) and water (1 ml) before adding LiOH (47 mg, 1.96 mmol). The heterogeneous mixture turned homogenous upon stirring at room temperature for 1 hour. Addition of aqueous 2N HCl and water induced formation of a precipitate which was filtered and washed with water. Compound 23 was isolated as a white solid (49 mg, 48% yield). The NMR and mass spectra results for 4-(2-benzoylamino-ethylsulfanyl)-pyridine-2,6-dicarboxylic acid (compound 23) were: ¹H NMR (300 MHz, DMSO-d₆): δ 3.44 (t, J=6.4, 2H), 3.58 (q, J=5.9, 2H), 7.45 (t, J=7.0, 2H), 7.53 (t, J=7.0, 1H), 7.81 (d, J=7.3, 2H), 8.07 (s, 2H), 8.75 (t, J=4.9, 1H); MS m/z 347(M+1).

For the synthesis of 4-{2-[(3,5-dihydroxy-naphthalene-2-carbonyl)-amino]-ethylsulfanyl}-pyridine-2,6-dicarboxylic acid (compound 24; FIG. 7), compound 4 (HCl salt) (80 mg, 0.261 mmol), 3,5-dihydroxy-2-naphthalenecarboxylic acid (53 mg, 0.260 mmol) and HOBt.H₂O (48 mg, 0.313 mg) were dissolved in DMF (1 ml). After adding triethylamine (0.11 ml, 0.791 mmol) and EDCI (60 mg, 0.313 mmol), the mixture was stirred at room temperature for 2.5 days. Addition of aqueous 2N HCl induced formation of an orange precipitate (39 mg) that was collected and washed with aqueous 2N HCl. 37 mg of that product was mixed with water (0.5 ml) and LiOH (15 mg, 0.62 mmol) and stirred at room temperature for 1.5 hours. After adding aqueous 2N HCl, the reddish solid compound 24 was collected on a funnel, washed with water and dried in vacuo (28 mg, 27% yield). The NMR and mass spectra results for 4-{2-[(3,5-dihydroxy-naphthalene-2-carbonyl)-amino]-ethylsulfanyl}-pyridine-2,6-dicarboxylic acid (compound 24) were: ¹H NMR (300 MHz, DMSO-d₆): δ 3.47 (m, 2H), 3.68 (m, 2H), 6.86 (d, J=7.3, 1H), 7.13 (t, J=7.8, 1H), 7.29 (d, J=8.2, 1H), 7.45 (s, 1H), 8.12 (s, 2H), 8.35 (s, 1H), 9.23 (br. t., 1H), 10.10 (br. s., 1H) and 10.60 (br. s., 1H) 2×OH; MS m/z 385 (M+H−CO₂).

### EXAMPLE VI

### Assay Methods for Various Oxidoreductases

This example describes assay protocols used to test binding and/or inhibitory activity of common ligands or bi-ligands.

Compounds were screened for binding to one or more of the following enzymes: dihydrodipicolinate reductase (DHPR), lactate dehydrogenase (LDH), alcohol dehydrogenase (ADH), dihydrofolate reductase (DHFR), 1-deoxy-D-xylulose-5-phosphate reductase (DOXPR), glyceraldehyde-3-phosphate dehydrogenase (GAPDH), 3-isopropylmalate dehydrogenase (IPMDH), inosine-5′-monophosphate dehydrogenase (IMPDH), aldose reductase (AR), and HMG CoA reductase (HMGCoAR).

Dihydrodipicolinate Reductase (DHPR)

For DHPR analysis, the compounds were screened using a kinetic protocol that spectrophotometrically evaluates oxidation of NADPH. Stock solutions of each of the reagents were prepared in the following concentrations. Dilutions of the stock solutions were prepared prior to running the assay in the concentrations indicated below. DHPR was diluted in 10 mM HEPES, pH 7.4. Dihydrodipicolinate synthase (DHPS) was not diluted and was stored in eppendorf tubes.

| | Stock | | Final | | Volume needed | |
|---|---|---|---|---|---|---|
| ddH₂O | | | | | 798 | μl |
| HEPES (pH 7.8) | 1 | M | 0.1 | M | 100 | μl |
| Pyruvate | 50 | mM | 1 | mM | 20 | μl |
| NADPH | 1 | mM | 6 | μM | 6 | μl |
| L-ASA | 28.8 | mM | 40 | μM | 13.9 | μl |
| DHPS | 1 | mg/ml | | | 7 | μl |
| DHPR | 1:1000 dilution of | | | | 5 | μl |
| | 1 mg/ml stock | | | | | |
| Inhibitor | 15 | mM | 100 | μM | 6.7 | μl |
| | | | | | (0.67% | DMSO) |
| DMSO | 100% | | ˜5% | | 43.3 | μl |
| or | | | | | | |
| Inhibitor | 10 | μM | 500 | nM | 50 | μl |
| | | | | | (5% | DMSO) |
| DMSO | 100% | | ˜5% | | 0 | μl |

Total Assay volume = 1000 μl

The L-aspartate semialdehyde (L-ASA) solution was prepared in the following manner. A 180 μM stock solution of ASA was prepared. 100 μl of the ASA stock solution was mixed with 150 μl of concentrated NaHCO₃ and 375 μl of H₂O. This mixture of reagents resulted in a 28.8 mM L-ASA stock solution >625 μl. The L-ASA stock solution was kept at a temperature of −20° C. After dilution, the pH of the 28.8 mM solution was checked and maintained between pH 1 to 2.

The DHPS reaction was monitored at 340 nm prior to and after addition of the inhibitor to detect background reaction with the inhibitor. The solution for background detection was a 945 μl solution containing 0.1 M HEPES, pH 7.8, 1 mM pyruvate, 6 μM NADPH, 40 μM L-ASA, and 7 μl of 1 mg/ml DHPS at 25° C. in the volumes indicated above. The sample solution was then mixed and incubated for 10 minutes. Inhibitor solutions (500 nM or 100 μM final concentration) and enough dimethylsulfoxide (DMSO) to provide a final DMSO concentration of 5% were added. The solution was mixed and incubated for an additional 6 minutes.

In DHPR samples, the DHPR stock solution was diluted and 5 μl of diluted E. coli DHPR enzyme (1:1000 dilution of 1 mg/ml stock) were added. The sample was mixed for 20 seconds, and then the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 2.58 mM was substituted for inhibitor to yield 70 to 80% inhibition.

For assaying bi-ligands, the substrate concentration and the NADPH or NADH concentration were kept near their Km values. When common ligands (CLMs) were measured, the substrate concentration was increased to at least 10 times the Km (1 mM L-ASA). If substrate mimics (specificity ligands) were tested, the NADPH or NADH concentration was increased.

Lactate Dehydrogenase (LDH)

For LDH analysis, the compounds were screened using a kinetic protocol that spectrophotometrically evaluates oxidation of NADH. Stock solutions of each of the reagents were prepared in the following concentrations. Dilutions of the stock solutions were prepared prior to running the assay in the concentrations indicated below.

| | Stock | | Final | | Volume needed | |
|---|---|---|---|---|---|---|
| ddH₂O | | | | | 780 | μl |
| HEPES (pH 7.4) | 1.0 | M | 0.1 | M | 100 | μl |
| Pyruvate | 50 | mM | 2.5 | mM | 50 | μl |
| NADH | 1 | mM | 10 | μM | 10 | μl |
| LDH | 1:2000 dilution of | | | | 10 | μl |
| | 1 mg/ml stock | | | | | |
| Inhibitor | 15 | mM | 100 | μM | 6.7 | μl |
| | | | | | (0.67% | DMSO) |
| DMSO | 100% | | 5% | | 43.3 | μl |

Total Assay volume = 1000 μl

The LDH reaction was monitored at 340 nm prior to and after addition of the inhibitor to detect background reaction with the inhibitor. Inhibitor solutions were prepared in DMSO so that, when diluted, the inhibitor concentration was 100 μM and the final DMSO concentration was adjusted to 5%. These solutions were incubated for 6 minutes at 25° C. in 990 μl of a solution containing 0.1 M HEPES, pH 7.4, 10 mM NADH, and 2.5 mM of pyruvate. The reaction was then initiated with 10 μl of diluted LDH from Rabbit Muscle (0.5 μg/ml; 1:2000 dilution of 1.0 mg/ml stock solution). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 10.3 mM was substituted for inhibitor to yield 50 to 70% inhibition.

When bi-ligands were screened, the substrate concentration and NADPH or NADH concentrations were kept near their Km values. When assaying common ligands (CLMs), the substrate concentration was increased to at least 10 times the Km (final concentration of pyruvate was about 2.5 mM). If the compounds assayed were specificity ligands (substrate mimics), the NAPDH or NADH concentration was increased.

Alcohol Dehydrogenase (ADH)

For ADH analysis, the compounds were screened using a kinetic protocol that spectrophotometrically evaluates reduction of NAD+.

Stock solutions of each of the reagents were prepared in the following concentrations. Dilutions of the stock solutions were prepared prior to running the assay in the concentrations indicated below.

| | Stock | | Final | | Volume needed | |
|---|---|---|---|---|---|---|
| ddH₂O | | | | | 787 | μl |
| HEPES (pH 8.0) | 1 | M | 0.1 | M | 100 | μl |
| EtOH | 10 | M | 130 | mN | 13 | μl |
| NAD+ | 2 | mM | 80 | μM | 40 | μl |
| ADH | 1:400 dilution of | | | | 10 | μl |
| | 1 mg/ml stock | | | | | |
| Inhibitor | 15 | mM | 100 | μM | 6.7 | μl |
| | | | | | (0.67% | DMSO) |
| DMSO | 100% | | 5% | | 43.3 | μl |

Total Assay volume = 1000 μl

The ADH reaction was monitored at 340 nm prior to and after addition of the inhibitor to detect background reaction with the inhibitor. Inhibitor solutions were prepared in DMSO so that, when diluted, the inhibitor concentration was 100 μM and the final DMSO concentration was adjusted to 5%. These solutions were incubated for 6 minutes at 25° C. in a 990 μl of a solution containing 0.1 M HEPES, pH 8.0, 80 μM NAD+, and 130 mM of ethanol. The reaction was then initiated with 10 μl of ADH from Bakers Yeast (Saccharomyces cerevisiae) (3.3 μg/ml; 1:400 dilution of 1.0 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 15.5 μM was substituted for inhibitor to yield 50 to 60% inhibition.

Where only a simple read was desired, as in the case of NAD+ concentration determination, 13 μl (10 M stock) of ethanol was used to drive the reaction, and 10 μl of pure enzyme (1 mg/ml) was used. NAD+ was soluble at 2 mM. In this situation, the procedure was as follows. All of the ingredients except for the enzyme were mixed together. The solution was mixed well and the absorbance at 340 nm read. The enzyme was added and read again at OD 340 after the absorbance stopped changing, generally 10 to 15 minutes after the enzyme was added.

Dihydrofolate Reductase (DHFR)

For DHFR analysis, the compounds were screened using a kinetic protocol that spectrophotometrically evaluates oxidation of NADH. Stock solutions of each of the reagents were prepared in the following concentrations. Dilutions of the stock solutions were prepared prior to running the assay in the concentrations indicated below. H2 folate was dissolved in DMSO to about 10 mM and then diluted with water to a concentration of 0.1 mM.

| | Stock | | Final | | Volume needed | |
|---|---|---|---|---|---|---|
| ddH₂O | | | | | 616 | μl |
| Tris-HCl (pH 7.0) | 1 | M | 0.1 | M | 100 | μl |
| KCl | 1 | mM | 0.15 | M | 150 | μl |
| H2 Folate | 0.1 | mM | 5 | μM | 50 | μl |
| NADPH | 2 | mM | 52 | μM | 26 | μl |
| DHFR | 1:85 dilution of | | | | 8 | μl |
| | 4 mg/ml stock | | | | | |
| Inhibitor | 15 | mM | 100 | μM | 6.7 | μl |
| DMSO | 100% | | 5% | | 43.3 | μl |

Total Assay volume = 1000 μl

The DHFR reaction was monitored at 340 nm prior to and after addition of the inhibitor to detect background reaction with the inhibitor. Inhibitor solutions were prepared in DMSO so that, when diluted, the inhibitor concentration was 100 μM and the final DMSO concentration was adjusted to 5%. These solutions were incubated for 6 minutes at 25° C. in a 992 μl of a solution containing 0.1 M Tris-HCl, PH 7.0, 150 mM KCl, 5 μM H2 folate, and 52 μM NADH. The reaction was then initiated with 8 μl of human DHFR (0.047 mg/ml; 1:85 dilution of 4 mg/ml stock solution). After the enzyme was added, the reaction was run for 10 minutes. Cuvette #1 contained the control reaction (no inhibitor), and cuvette #2 contained the positive control reaction in which Cibacron Blue at 3 μM was substituted for inhibitor to yield 50 to 70% inhibition.

When bi-ligands were screened, the substrate concentration and NADPH or NADH concentrations were kept near their Km values. When assaying common ligands (CLMs), the substrate concentration was increased to at least 10 times the Km. If the compounds assayed were specificity ligands (substrate mimics), the NAPDH or NADH concentration was increased.

1-Deoxy-D-xylulose-5-phosphate Reductoisomerase (DOXPR)

For DOXPR analysis, the compounds were screened using a kinetic protocol that spectrophotometrically evaluates oxidation of NADPH. Stock solutions of each of the reagents were prepared in the following concentrations. Dilutions of the stock solutions were prepared prior to running the assay in the concentrations indicated below. DOXPR was diluted in 10 mM HEPES, pH 7.4.

| | Stock | | Final | | Volume needed | |
|---|---|---|---|---|---|---|
| ddH₂O | | | | | 707 | μl |
| HEPES (pH 7.4) | 1 | M | 0.1 | M | 100 | μl |
| DOXP | 10 | mM | 1.15 | mM | 115 | μl |
| NADPH | 1 | mM | 8 | μM | 8 | μl |
| MnCl₂ | 100 | mM | 1 | mM | 10 | μl |
| DOXPR | 1:200 dilution of | | | | 10 | μl |
| | 2 mg/ml stock | | | | | |
| Inhibitor | 15 | mM | 100 | μM | 6.7 | μl |
| | | | | | (0.67% | DMSO) |
| DMSO | 100% | | 5% | | 43.3 | μl |

Total Assay volume = 1000 μl

The DOXPR reaction was monitored at 340 nm prior to and after addition of the inhibitor to detect background reaction with the inhibitor. Solutions of the inhibitors in DMSO were prepared to provide a final DMSO concentration of 5%. These solutions were incubated for 6 minutes at 25° C. in a 990 μl of a solution containing 0.1 M HEPES, pH 7.4, 1 mM MnCl₂. 1.15 mM DOXP, and 8 μM NADPH. The reaction was then initiated with 10 μl of DOXP reductoisomerase (also known as DOXP reductase) from E. coli (10 μg/ml; 1:200 diilution of 2 mg/ml stock solution). After the enzyme was added, the reaction was run for 10 minutes, and the samples were read in a Cary spectrophotometer at 340 nm. Cuvette #1 contained the control reaction (no inhibitor), and cuvette #2 contained the positive control reaction in which Cibacron Blue at 10.32 μM was substituted for inhibitor to yield 70 to 80% inhibition.

When bi-ligands were screened, the substrate concentration and NADPH or NADH concentrations were kept near their Km values. When assaying common ligands (CLMs), the substrate concentration was increased to at least 10 times the Km. If the compounds assayed were specificity ligands (substrate mimics), the NAPDH or NADH concentration was increased.

Glyceraldehyde-3-phosphate Dehydrogenase (GAPDH)

For GAPDH analysis, the compounds were screened using a kinetic protocol that spectrophotometrically evaluates reduction of NAD+. Stock solutions of each of the reagents were prepared in the following concentrations. Dilutions of the stock solutions were prepared prior to running the assay in the concentrations indicated below.

| | Stock | | Final | | Volume needed | |
|---|---|---|---|---|---|---|
| ddH₂O | | | | | 739 | μl |
| Triethanolamine | 1 | M | 25 | mM | 125 | μl |
| (pH 7.5) | | | | | | |
| GAP | 50 | mM | 145 | μM | 3 | μl |
| NAD+ | 5 | mM | 0.211 | mM | 42 | μl |
| Sodium Arsenate | 200 | mM | 5 | mM | 25 | μl |
| βME | 500 | mM | 3 | mM | 6 | μl |
| GAPDH | 1:200 dilution of | | | | 10 | μl |
| | 1 mg/ml stock | | | | | |
| Inhibitor | 12.5 | mM | 100 | μM | 8 | μl |
| | | | | | (total 5% | DMSO) |
| DMSO | 100% | 5% | 42 | μl | | |

Total Assay volume = 1000 μl

The GAPDH reaction was monitored at 340 nm prior to and after addition of the inhibitor to detect background reaction with the inhibitor. Inhibitors were incubated for 6 minutes at 25° C. in a 990 μl of a solution containing 125 mM triethanolamine, pH 7.5, 145 μM glyceraldehyde 3-phosphate (GAP), 0.211 mM NAD, 5 mM sodium arsenate, and 3 mM β-metcaptoethanol (βME). The reaction was then initiated with 10 μl of E. coli GAPDH (1:200 dilution of 1.0 mg/ml stock solution). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. The final concentration of DMSO in a cuvette was about 5%. Cuvette #1 contained the control reaction (no inhibitor).

For use in these experiments, GAP was deprotected from diethyl acetal in the following manner. Water was boiled in a recrystallizing dish. Dowex (1.5 mg) and GAP (200 mg; SIGMA G-5376; Sigma, St. Louis Mo.) were weighed and placed in a 15 ml conical tube. The Dowex and GAP were resuspended in 2 ml dH₂O, followed by shaking of the tube until the GAP dissolved. The tube was then immersed, while shaking, in boiling water for 3 minutes. The tube was placed in an ice bath to cool for 5 minutes. As the sample cooled, the resin settled to the bottom of the test tube, allowing removal of the supernatant with a pasteur pipette. The supernatant was filtered through a 0.45 or 0.2 μM cellulose acetate syringe filter. The filtered supernatant was retained, and another 1 ml of dH₂O was added to the resin tube. The tube was then shaken and centrifuged for 5 minutes at 3,000 rpm. The supernatant was again removed with a pasteur pipette and passed through a 0.45 or 0.2 mM cellulose acetate syringe filter. The two supernatant aliquots were then pooled to provide a total GAP concentration of about 50 mM. The GAP was then divided into 100 μl aliquots and stored at −20° C. until use.

Inosine-5′-monophosphate Dehydrogenase (IMPDH)

For IMPDH analysis, the compounds were screened using a kinetic protocol that spectrophotometrically evaluates reduction of NAD+. Stock solutions of each of the reagents were prepared in the following concentrations. Dilutions of the stock solutions were prepared prior to running the assay in the concentrations indicated below.

| | Stock | | Final | | Volume needed | |
|---|---|---|---|---|---|---|
| ddH₂O | | | | | 447 | μl |
| Tris-HCl (pH 8.0) | 1 | M | 0.1 | M | 100 | μl |
| KCl | 1 | M | 0.25 | M | 250 | μl |
| NAD+ | 2 | mM | 30 | μM | 15 | μl |
| IMP | 6 | mM | 600 | μM | 100 | μl |
| Glycerol | 10% | | 0.3% | | 30 | μl |
| IMPDH | 0.75 mg/ml (undiluted) | | | | 8 | μl |
| Inhibitor | 15 | mM | 100 | μM | 6.7 | μl |
| | | | | | (0.67% | DMSO) |
| DMSO | 100% | | 5% | | 43.3 | μl |

Total Assay volume = 1000 μl

The reaction human inosine monphosphate dehydrogenase II (IMPDH) was monitored at 340 nm prior to and after addition of the inhibitor to detect background reaction with the inhibitor. Solutions of the inhibitors in DMSO were prepared to provide a final DMSO concentration of 5%. These solutions were incubated for 6 minutes at 37° C. in a 992 μl of a solution containing 0.1 M Tris-HCl, PH 8.0, 0.25 M KCl, 0.3% glycerol, 30 μM NAD+, and 600 μM inosine monophosphate (IMP). The reaction was then initiated with 8 μl of human IMPDH (0.75 μg/ml). After the enzyme was added, the reaction was run for 10 minutes and read in a Cary spectrophotometer at 340 nm. Cuvette #1 contained the control reaction (no inhibitor), and cuvette #2 contained the positive control reaction in which Cibacron Blue was substituted for inhibitor.

HMG CoA Reductase (HMGCoAR)

For HMGCoAR analysis, the compounds were screened using a kinetic protocol that spectrophotometrically evaluates oxidation of NADPH. Stock solutions of each of the reagents were prepared in the following concentrations. Dilutions of the stock solutions were prepared prior to running the assay in the concentrations indicated below. The enzyme was diluted in 1 M NaCl. To prepare the dilution buffer, 10 μl of HMGCoAR (1 mg/ml) was mixed with 133 μl of 3 M NaCl solution and 257 μl of 25 mM KH₂PO₄ buffer, pH 7.5, containing 50 mM NaCl, μl mM ethylenediaminetetraacetic acid EDTA, and 5 mM dithiothreitol (DTT).

| | Stock | | Final | | Volume needed | |
|---|---|---|---|---|---|---|
| ddH2O | | | | | 841 | μl |
| KH₂PO₄ (pH 7.5) | 1 | M | 25 | mM | 25 | μl |
| HMGCoA | 10 | mM | 160 | μM | 16 | μl |
| NADPH | 1 | mM | 13 | μM | 13 | μl |
| NaCl | 1 | M | 50 | mM | 50 | μl |
| EDTA | 50 | mM | 1 | mM | 20 | μl |
| DTT | 500 | mM | 5 | mM | 10 | μl |
| HMGCoAR | 1:40 dilution of | | | | 5 | μl |
| | 0.65 mg/ml stock | | | | | |
| Inhibitor | 10 | mM | 100 | μM | 10 | μl |
| DMSO | 100% | | ˜2% | | 10 | μl |

Total Assay volume = 1000 μl

The reaction of HMGCOAR was Staphylococcus aureus monitored at 340 nm prior to and after addition of the inhibitor to detect background reaction with the inhibitor. Solutions of 100 μM of the inhibitors in DMSO were prepared to provide a final DMSO concentration of 2%. These solutions were incubated for 6 minutes at 25° C. in a 994 μl of a solution containing 25 mM KH₂PO₄, pH 7.5, 160 μM HMGCoA, 13 μM NADPH, 50 mM NaCl, 1 mM EDTA, and 5 mM DTT. The reaction was initiated with 5 μL of HMGCoAR enzyme from Staphylococcus aureus (1:40 final dilution of 0.65 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 2.05 μM was substituted for inhibitor to yield 50 to 70% inhibition.

When bi-ligands were screened, the substrate concentration and NADPH or NADH concentrations were kept near their Km values. When assaying common ligands (CLMs), the substrate concentration was increased to at least 10 times the Km. If the compounds assayed were specificity ligands (substrate mimics), the NAPDH or NADH concentration was increased.

3-Isopropylmalate Dehydrogenase (IPMDH)

For IPMDH analysis, the compounds were screened using a kinetic protocol that spectrophotometrically evaluates reduction of NAD. Stock solutions of each of the reagents were prepared in the following concentrations. Dilutions of the stock solutions were prepared prior to running the assay in the concentrations indicated below.

| | Stock | | Final | | Volume needed | |
|---|---|---|---|---|---|---|
| ddH₂O | | | | | 407 | μl |
| KH₂PO_{4 (pH 7.6)} | 1 | M | 20 | mM | 20 | μl |
| KCl | 1 | M | 0.3 | M | 300 | μl |
| MnCl₂ | 20 | mM | 0.2 | mM | 10 | μl |
| NAD | 3.3 | mM | 109 | μM | 33 | μl |
| IPM | 2 | mM | 340 | μM | 170 | μl |
| IPMDH | 1:300 dilution of | | | | 10 | μl |
| | 2.57 mg/ml stock | | | | | |
| Inhibitor | 16 | mM | 200 | μM | 12.5 | μl |
| DMSO | 100% | | 5% | | 37.5 | μl |

Total Assay volume = 1000 μl

The reaction of Escherichia coli IPMDH was monitored at 340 nm prior to and after addition of the inhibitor to detect background reaction with the inhibitor. Inhibitor was incubated for 5 minutes at 37° C. in a 990 μl of a solution containing 20 mM potassium phosphate, pH 7.6, 0.3 M potassium chloride, 0.2 mM manganese chloride, 109 μM NAD, and 340 μM DL-threo-3-isopropylmalic acid (IPM). The reaction was then initiated with 10 μl of E. coli isopropylmalate dehydrogenase (1:300 dilution of 2.57 mg/ml stock solution). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. The final concentration of DMSO in the cuvette was 5%. Cuvette #1 contained the control reaction (no inhibitor), and cuvette #2 contained the positive control reaction in which Cibacron Blue was substituted for inhibitor to yield 30 to 70% inhibition.

Aldose Reductase (AR)

For AR analysis, the compounds were screened using a kinetic protocol that spectrophotometrically measures enzyme activity. Stock solutions of each of the reagents were prepared in the following concentrations. Dilutions of the stock solutions were prepared prior to running the assay in the concentrations indicated below.

| | | | | | Volume | |
|---|---|---|---|---|---|---|
| | Stock | | Final | | needed | |
| ddH₂O | | | | | 565.5 | μl |
| KH₂PO₄ (pH 7.5) | 1 | M | 100 | mM | 100 | μl |
| Ammonium Sulfate | 1 | M | 0.3 | M | 300 | μl |
| EDTA | 500 | mM | 1 | mM | 2 | μl |
| NADPH | 1 | mM | 3.8 | μM | 3.8 | μl |
| Glyceraldehyde | 100 | mM | 171 | μM | 1.7 | μl |
| DTT | 100 | mM | 0.1 | mM | 1 | μl |
| Human ALDR | 1:5 dilution of | | | | 10 | μl |
| | 0.55 mg/ml stock | | | | | |
| Inhibitor | 12.5 | mM | 200 | μM | 16 | μl |

Total Assay volume = 1000 μl

The AR reaction was monitored at 340 nm prior to and after addition of the inhibitor to detect background reaction with the inhibitor. Inhibitor solutions were incubated for 5 minutes at 25° C. in a 990 μl of a solution containing 100 mM potassium phosphate, pH 7.5, 0.3 M ammonium sulfate, 1.0 mM ethylenediaminetetraacetic acid (EDTA), 3.8 mM B-Nicotinamide adenine dinucleotide phosphate (NADPH), 171 μM DL-glyceraldehyde and 0.1 mM DL-dithiothreitol. The reaction was then initiated with 10 μl of human Aldose Reductase (1:5 dilution of 0.55 mg/ml stock solution). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. The final DMSO concentration in the cuvette was 5%. Cuvette #1 contained the control reaction (no inhibitor), and cuvette #2 contained the positive control reaction in which Cibacron Blue was substituted for inhibitor to yield 30 to 70% inhibition.

This example describes various assay protocols used to screen for inhibitor activity of various bi-ligands.

### EXAMPLE VII

### Screening of Biligands for Binding to Dihydrodipicolinate Reductase (DHPR)

This example describes the screening of bi-ligands having variant common ligands for binding activity to dihydrodipicolinate reductase (DHPR).

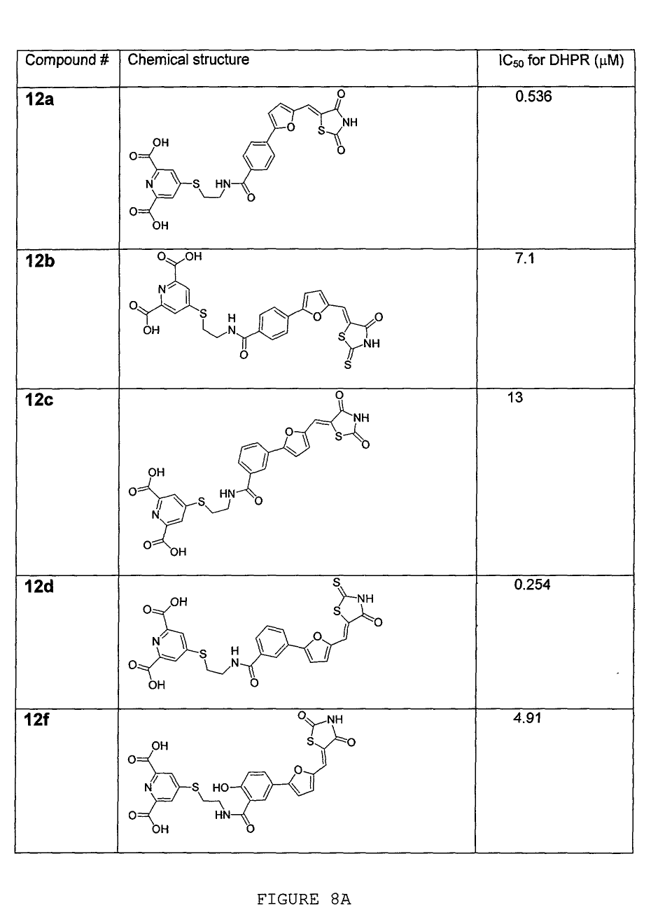

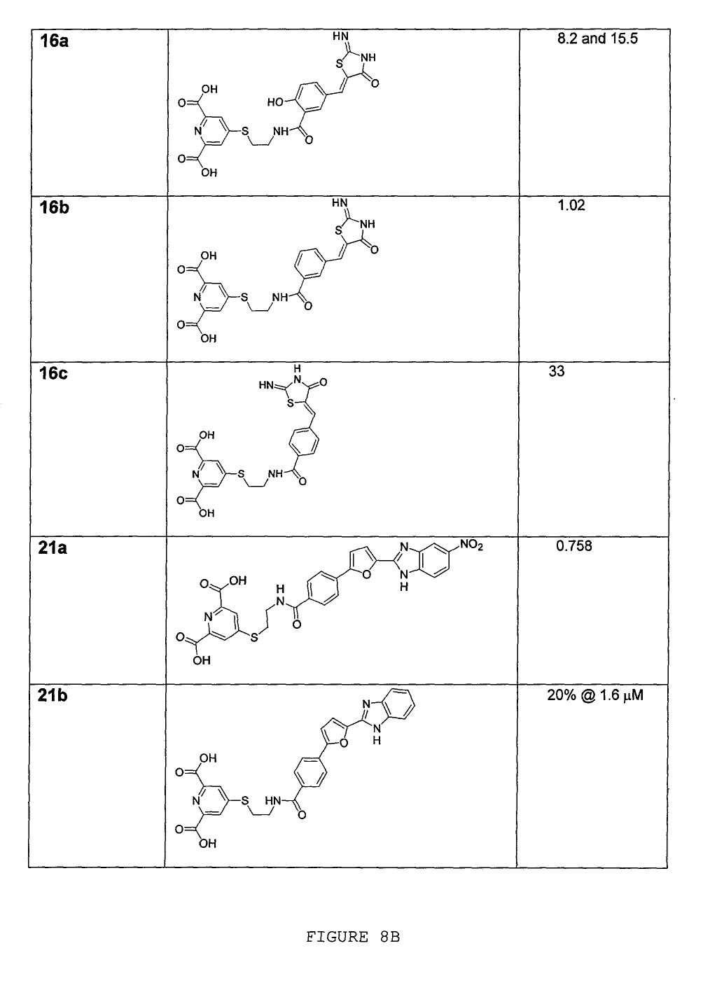

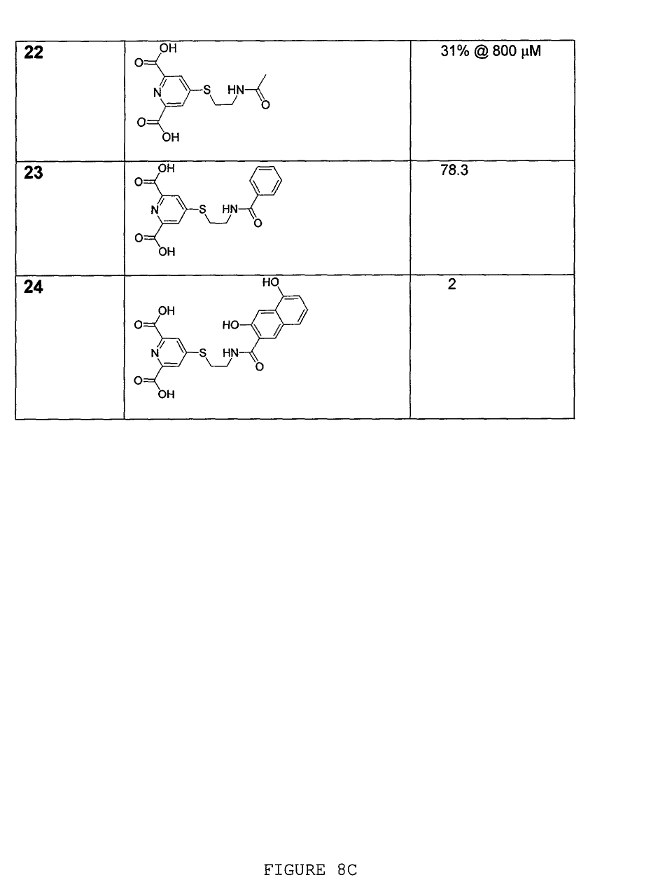

Ligands synthesized as described in Examples II-IV were screened for binding to DHPR essentially as described in Example VI. IC₅₀ data for these compounds are presented in FIG. 8.

The rhodanine and thiazolidinedione derivative biligands 12a, 12b, 12c, 12d and 12f displayed IC₅₀ values for dihydrodipicolinate reductase (DHPR) of about 0.536 μM, 7.1 μM, 13 μM, 0.254 μM, and 4.91 μM, respectively (FIG. 8A). The pseudothiohydantoin derivative biligands 16a, 16b and 16c displayed IC₅₀ values of about 8.2 and 15.5 μM, 1.02 μM, and 33 μM, respectively (FIG. 8B). The benzimidazole biligand 21a displayed an IC₅₀ value of about 0.758 μM. The benzimidazole biligand 21b displayed about 20% inhibition of DHFR activity at about 1.6 μM. The pyridine dicarboxylate derivative 22 displayed about 31% inhibition of DHFR activity at about 800 μM. The pyridine dicarboxylate derivatives 23 and 24 displayed IC₅₀ values of 78.3 μM and 2 μM respectively.

These results show the inhibitory activity of various bi-ligands for dihydrodipicolinate reductase.

### EXAMPLE VIII

### Binding Activity of Rhodanine Derivatives

This example describes the screening of rhodanine derivatives as common ligand variants for binding to a receptor in a receptor family.

Rhodanine derivatives were synthesized as described in Example II. The derivatives were screened for binding activity to DHPR essentially as described in Example VI.

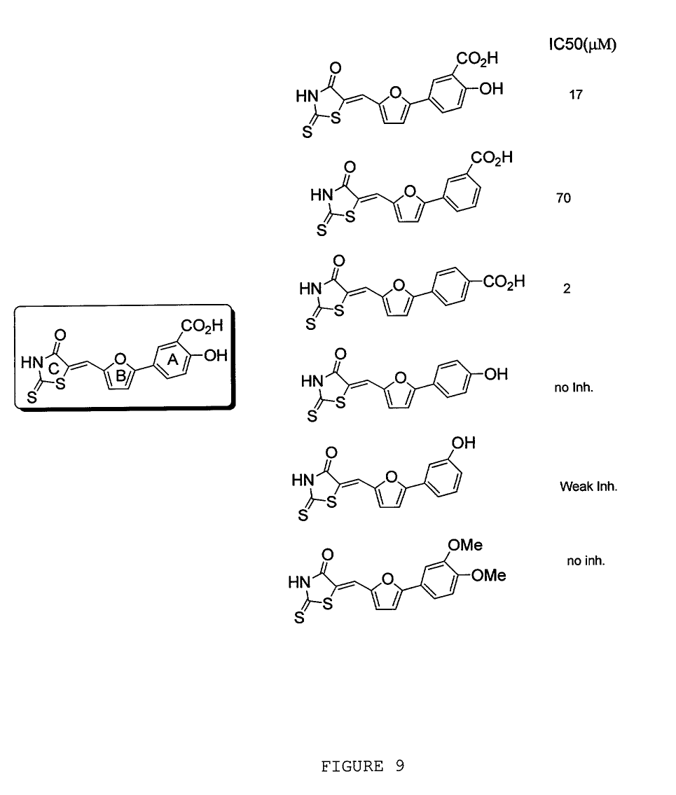

The resulting IC₅₀ data for 6 exemplary rhodanine derivatives are presented in FIG. 9. While three of the derivatives showed weak or no inhibition, three rhodanine derivatives were identified as having an IC₅₀ of about 2 μM to about 70 μM.

These results show the inhibitory activity of various rhodanine derivatives.

### EXAMPLE IX

### Binding Activity of Thiazolidinedione Derivatives

This example describes the screening of thiazolidinedione derivatives as common ligand variants for binding to a receptor in a receptor family.

Thiazolidine derivatives were synthesized as described in Example II. The derivatives were screened for binding activity to DHPR essentially as described in Example VI.

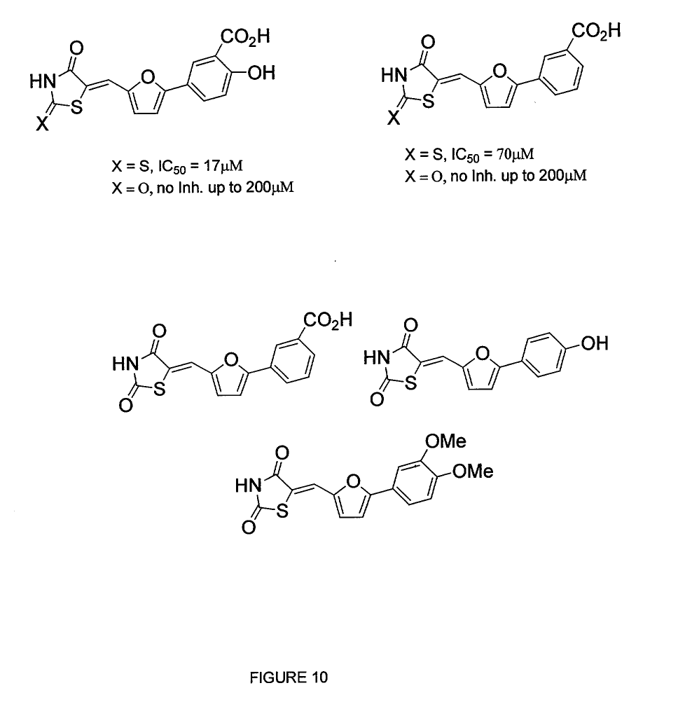

The binding data for 5 exemplary thiazolidinedione derivatives are presented in FIG. 10. Three of the derivatives exhibited no inhibition (bottom 3 compounds shown in FIG. 10). Two of the thiazolidinedione analogs were identified as having Ki values of about 17 μM and about 79 μM, respectively, for two derivatives having sulfur at the “X” position, but no inhibition when “X” is oxygen.

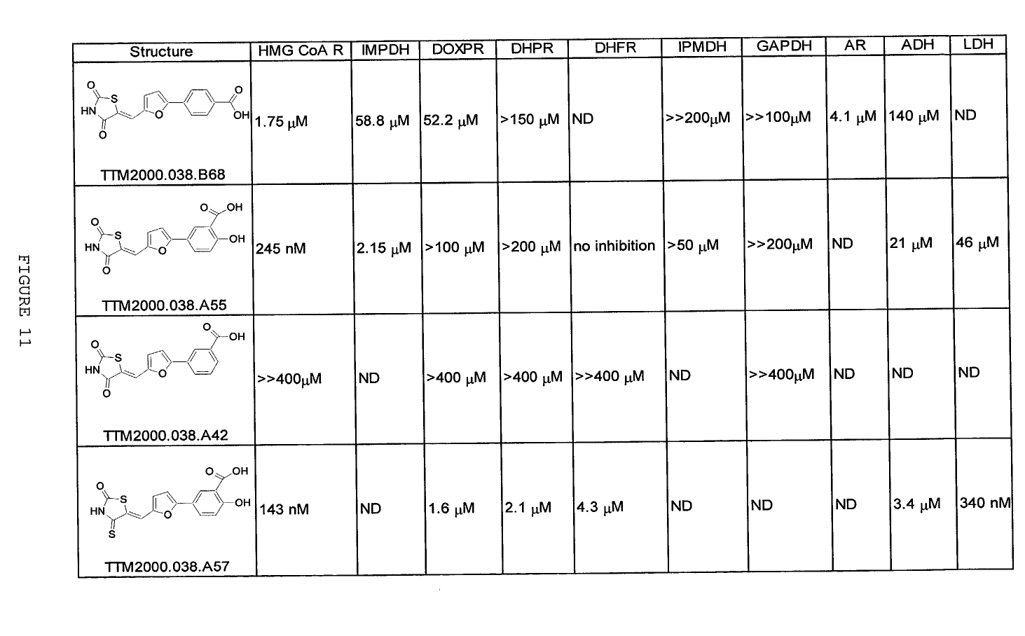

The activity of various thiazolidinedione analogs to various members of an oxidoreductase receptor family is shown in FIG. 11. Representative thiazolidinedione common ligand mimics were selected for characterization of binding activity. The binding activity was tested against hydroxymethylglutaryl coenzyme A (HMG-CoA) reductase, inosine monophosphate dehydrogenase (IMPDH), 1-deoxy-D-xylulose-5-phospate reductase (DOXPR), dihydrodipicolinate reductase (DHPR), dihydrofolate reductase (DHFR), isopropylmalate dehydrogenase (IPMDH), glyceraldehyde-3-dehydrogenase (GAPDH), aldose reductase (AR), alcohol dehydrogenase (ADH) and lactate dehydrogenase (LDH). The enzymes were assayed essentially as described in Example VI.

As shown in FIG. 11, the thiazolidinedione common ligand mimic TTM2000.038.B68 displayed IC₅₀ values of 1.75 μM and 49.3 μM for HMGCoAR, 58.8 μM for IMPDH, 52.2 μM for DOXPR, >150 μM for DHPR, 4.1 μM and 2.3 μM for AR, and 140 μM and 116 μM for ADH. The thiazolidinedione common ligand mimic TTM2000.038.A55 displayed IC₅₀ values of 245 nM for HMGCOAR, 2.15 μM for IMPDH, >100 μM for DOXPR, >200 μM for DHPR, >50 μM for IPMDH, 21 μM for ADH, and 46 μM for LDH. The thiazolidinedione common ligand mimic TTM2000.038.A42 displayed >400 μM for DOXPR and >400 μM for DHPR. The thiazolidinedione common ligand mimic TTM2000.038.A57 displayed IC50 values of 143 nM for HMGCOAR, 1.6 μM for DOXPR, 2.1 μM for DHPR, 4.3 μM for DHFR, 3.4 μM for ADH and 340 nM for LDH. These results show that various thiazolidinediones have activity for various oxidoreductases.

These results indicate that the thiazolidinedione common ligand variants exhibit differential binding activity to various oxidoreductases.

### EXAMPLE X

### Binding Activity of Pseudothiohydantoin Derivatives

This example describes the screening of pseudothiohydantoin derivatives as common ligand variants for binding to a receptor in a receptor family.

Pseudothiohydantoin derivatives were synthesized as described in Example III. The derivatives were screened for binding activity to DOXPR, a member of an oxidoreductase receptor family, essentially as described in Example VI.

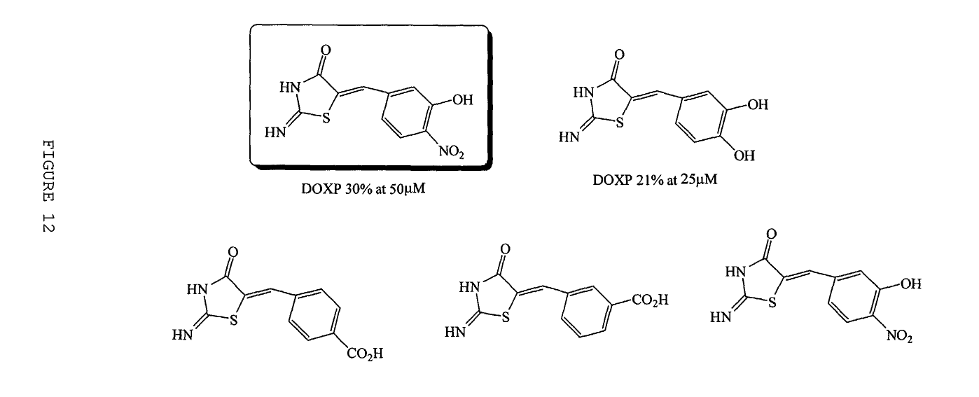

The binding data for 2 exemplary thiazolidinedione derivatives are presented in FIG. 12. The results shown for the two pseudothiohydantoin derivatives are for inhibition of 1-deoxy-D-xylulose 5-phospate reductoisomerase (DOXP). The derivatives displayed about 30% inhibition of DOXP at about 50 μM and about 21% inhibition of DOXP at about 25 μM, respectively. Also shown are two additional derivatives with a carboxylic acid at the ortho and para positions.

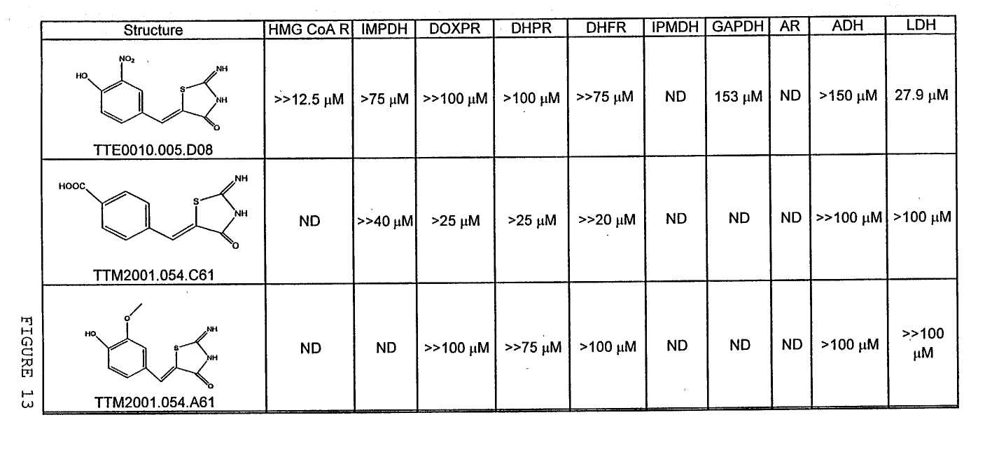

Pseudothiohydantoin derivatives were screened for binding to various oxidoreductases (see FIG. 13). As shown in FIG. 13, the pseudothiohydantoin common ligand mimic TTE0010.005.D08 displayed IC₅₀ values of >75 μM for IMPDH, >>100 μM for DOXPR, >100 μM for DHPR, 153 μM for GAPDH, >150 μM for ADH, and 27.9 μM for LDH. The pseudothiohydantoin common ligand mimic TTM2001.054.C61 displayed IC₅₀ values of >25 μM for DOXPR, >25 μM for DHPR, >>20 μM for DHFR, and >100 μM for LDH. The pseudothiohydantoin common ligand mimic TTM2001.054.A61 displayed >100 μM for DHFR and >100 μM for ADH. These results show that various pseudothiohydantoin have activity for various oxidoreductases.

The results shown indicate that the pseudothiohydantoin common ligand variants exhibited differential binding activity to various oxidoreductases.

### EXAMPLE XI

### Binding Activity of Benzimidazole Derivatives

This example describes the screening of benzimidazole derivatives as common ligand variants for binding to a receptor in a receptor family.

Benzimidazole derivatives were synthesized as described in Example IV. The derivatives were screened for binding activity to DHPR essentially as described in Example VI.

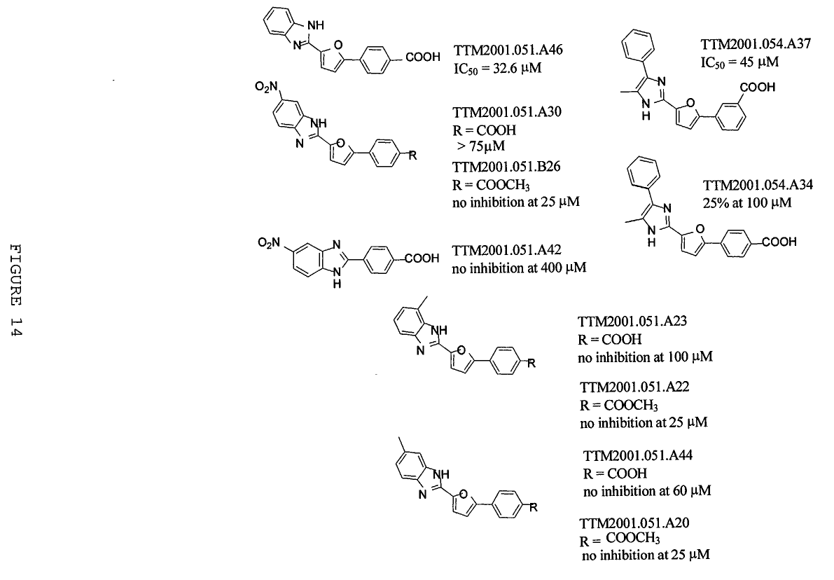

The binding activity for ten exemplary benzimidazole analogs are shown in FIG. 14. Three of the structures contain an R group, which is specified below the designation for the compound. While six of the analogs displayed no inhibition under the assay conditions used, two analogs, TTM2001.051.A46 and TTM2001.054.A37, displayed IC₅₀ values of about 32 μM and about 45 μM, respectively. A third analog, TTM2001.051.A30, displayed an IC₅₀ value of >75 μM, and a forth analog, TTM2001.054.A34, displayed an IC₅₀ value of >100 μM.

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

What is claimed is:

1. A method for identifying a common ligand for a receptor family, comprising the steps of:
(a) generating a population of molecules comprising a specificity ligand having binding activity for a receptor in a receptor family, said specificity ligand attached to a plurality of chemical moieties at a position on said specificity ligand to direct said specificity ligand to a specificity site and said chemical moieties to a conserved site of said receptor;
(b) screening said population of molecules for binding to said receptor; and
(c) identifying a bi-ligand having increased binding activity for said receptor relative to said specificity ligand alone, thereby identifying a common ligand having binding activity for said receptor.

2. The method of claim 1, wherein said specificity ligand is attached to an expansion linker, said expansion linker further attached to said plurality of chemical moieties and having sufficient length and orientation to direct said specificity ligand to a specificity site and said plurality of chemical moieties to a conserved site of said receptor.

3. The method of claim 1, wherein said receptor is an enzyme.

4. The method of claim 3, wherein the enzyme is selected from the group consisting of a kinase, oxidoreductase, GTPase, carboxyl transferase, acyl transferase, decarboxylase, transaminase, racemase, methyl transferase, formyl transferase, and α-ketodecarboxylase.

5. The method of claim 1, wherein said receptor family binds a cofactor selected from the group consisting of nicotinamide adenine dinucleotide (NAD), nicotinamide adenine dinucleotide phosphate (NADP), thiamine pyrophosphate, flavin adenine dinucleotide (FAD), flavin mononucleotide (FMN), pyridoxal phosphate, coenzyme A, tetrahydrofolate, adenosine triphosphate, guanosine triphosphate and S-adenosyl methionine (SAM).

6. A method of generating a population of bi-ligands to a receptor in a receptor family, comprising coupling a common ligand identified by the method of claim 1 to a plurality of chemical moieties at a position on said common ligand to direct said common ligand to a conserved site and said plurality of chemical moieties to a specificity site of a receptor in a receptor family.

7. The method of claim 6, wherein said common ligand is attached to an expansion linker, said expansion linker further attached to said plurality of chemical moieties and having sufficient length and orientation to direct said common ligand to a conserved site and said plurality of chemical moieties to a specificity site of said receptor.

8. The method of claim 6, wherein said receptor is an enzyme.

9. The method of claim 8, wherein the enzyme is selected from the group consisting of a kinase, oxidoreductase, GTPase, carboxyl transferase, acyl transferase, decarboxylase, transaminase, racemase, methyl transferase, formyl transferase, and α-ketodecarboxylase.

10. The method of claim 6, wherein said receptor family binds a cofactor selected from the group consisting of nicotinamide adenine dinucleotide (NAD), nicotinamide adenine dinucleotide phosphate (NADP), thiamine pyrophosphate, flavin adenine dinucleotide (FAD), flavin mononucleotide (FMN), pyridoxal phosphate, coenzyme A, tetrahydrofolate, adenosine triphosphate, guanosine triphosphate and S-adenosyl methionine (SAM).

11. The method of claim 6, wherein said population comprises bi-ligands having specificity for two or more receptors in a receptor family.

12. The method of claim 6, wherein said population comprises bi-ligands having specificity for three or more receptors in a receptor family.

13. A method of identifying a bi-ligand to a receptor in a receptor family, comprising:
(a) generating a population of molecules, said population comprising a common ligand identified by the method of claim 1, said common ligand attached to a plurality of chemical moieties at a position on said common ligand to direct said common ligand to a conserved site and said plurality of chemical moieties to a specificity site of a receptor in a receptor family;
(b) screening said population of molecules for binding to a receptor in said receptor family; and
(c) identifying a bi-ligand having binding activity and specificity for said receptor.

14. The method of claim 13, wherein steps (b) and (c) are repeated one or more times for another receptor in said receptor family.

15. The method of claim 13, wherein said common ligand is attached to an expansion linker, said expansion linker further attached to said plurality of chemical moieties and having sufficient length and orientation to direct said common ligand to a conserved site and said plurality of chemical moieties to a specificity site of said receptor.

16. The method of claim 13, wherein said receptor is an enzyme.

17. The method of claim 16, wherein the enzyme is selected from the group consisting of a kinase, oxidoreductase, GTPase, carboxyl transferase, acyl transferase, decarboxylase, transaminase, racemase, methyl transferase, formyl transferase, and α-ketodecarboxylase.

18. The method of claim 13, wherein said receptor family binds a cofactor selected from the group consisting of nicotinamide adenine dinucleotide (NAD), nicotinamide adenine dinucleotide phosphate (NADP), thiamine pyrophosphate, flavin adenine dinucleotide (FAD), flavin mononucleotide (FMN), pyridoxal phosphate, coenzyme A, tetrahydrofolate, adenosine triphosphate, guanosine triphosphate and S-adenosyl methionine (SAM).

19. The method of claim 13, wherein said population comprises bi-ligands having specificity for two or more receptors in a receptor family.

20. The method of claim 13, wherein said population comprises bi-ligands having specificity for three or more receptors in a receptor family.

21. A method of identifying a population of bi-ligands to receptors in a receptor family, comprising:
(a) generating a first population of molecules comprising a specificity ligand having binding activity for a receptor in a receptor family, said specificity ligand attached to a first plurality of chemical moieties at a position on said specificity ligand to direct said specificity ligand to a specificity site and said chemical moieties to a conserved site of said receptor;
(b) screening said population of molecules for binding to said receptor;
(c) identifying a bi-ligand having increased binding activity for said receptor relative to said specificity ligand alone, thereby identifying a common ligand having binding activity for said receptor;
(d) generating a second population of molecules, said population comprising said common ligand identified in step (c) attached to a second plurality of chemical moieties at a position on said common ligand to direct said common ligand to a conserved site and said plurality of chemical moieties to a specificity site of a receptor in a receptor family;
(e) screening said population of molecules for binding to a receptor in said receptor family;
(f) identifying a bi-ligand having binding activity and specificity for said receptor; and
(g) optionally repeating steps (e) and (f) one or more times for another receptor in said receptor family.

22. The method of claim 21, wherein said specificity ligand in said first population is attached to an expansion linker, said expansion linker further attached to said first plurality of chemical moieties and having sufficient length and orientation to direct said specificity ligand to a specificity site and said first plurality of chemical moieties to a conserved site of said receptor.

23. The method of claim 21, wherein said common ligand is attached to an expansion linker, said expansion linker further attached to said second plurality of chemical moieties and having sufficient length and orientation to direct said common ligand to a conserved site and said second plurality of chemical moieties to a specificity site of said receptor.

24. The method of claim 21, wherein said receptor is an enzyme.

25. The method of claim 24, wherein the enzyme is selected from the group consisting of a kinase, oxidoreductase, GTPase, carboxyl transferase, acyl transferase, decarboxylase, transaminase, racemase, methyl transferase, formyl transferase, and α-ketodecarboxylase.

26. The method of claim 21, wherein said receptor family binds a cofactor selected from the group consisting of nicotinamide adenine dinucleotide (NAD), nicotinamide adenine dinucleotide phosphate (NADP), thiamine pyrophosphate, flavin adenine dinucleotide (FAD), flavin mononucleotide (FMN), pyridoxal phosphate, coenzyme A, tetrahydrofolate, adenosine triphosphate, guanosine triphosphate and S-adenosyl methionine (SAM).

27. The method of claim 21, wherein said population comprises bi-ligands having specificity for two or more receptors in a receptor family.

28. The method of claim 21, wherein said population comprises bi-ligands having specificity for three or more receptors in a receptor family.
